SEQUENCE LISTING (72 sequences)

SEQ ID NO: 1 - DNA, 1023 bp, Zymomonas mobilis
atgaacacttctacgcaaaaacccgctcatttcgacaagatttcgatcaaagggattgataaatccgcaacccgtgtagcgttaggcacatgggctattggtggctggatgtggggcggcactgatgacgatgcctccattaaaaccattcatcgggcgattgatcttggtatcaatatcatcgacaccgctccggcttatggccgtggccatgctgaagaagtcgttggtaaagccatcaaaggtcaacgcgataatttgattattgcgaccaaagtcggccttgattggactttaacccccgaccaatcgatgcgccgtaacagttcagccagccgtatcaaaaaagaaatcgaagattctctgcgccgccttggcactgattatatcgacctttatcaggtgcattggccggatccgctggttccgattgaagaaaccgcaacaatattggaagccctcagaaaagaaggcaaaatccgttctatcggcgtttccaattattccgttcagcagatggacgagttcaagaaatatgccgagctggccgtttcgcagtcgccttataatctgtttgaacgcgaaatagacaaagacatcctgccctatgccaagaaaaacgatctggtcgttttaggctatggtgcgctttgccgtggtttactttctggcagaatgacggcggatcgtgcctttacaggcgatgatttacggaaaacagacccgaaattccagaaaccgcgctttgaacattatctggccgcggttgaagaactgaagaaactcgccaaagagcattacaataaatcggtgttggctttggctatccgctggatgttggagcaagggcccactttagcactttggggcgctcgcaagccggaacagatcgacggtattgatgaagtttttggctggcagatatcggatgaagatctgaaacagattgatgctattctggccaagaatatccccaatcctatcggtgcagaatttatggcacccccgccacgcgataaataa

SEQ ID NO: 2 - protein, 340 aa, Zymomonas mobilis
MNTSTQKPAHFDKISIKGIDKSATRVALGTWAIGGWMWGGTDDDASIKTIHRAIDLGINIIDTAPAYGRGHAEEVVGKAIKGQRDNLIIATKVGLDWTLTPDQSMRRNSSASRIKKEIEDSLRRLGTDYIDLYQVHWPDPLVPIEETATILEALRKEGKIRSIGVSNYSVQQMDEFKKYAELAVSQSPYNLFEREIDKDILPYAKKNDLVVLGYGALCRGLLSGRMTADRAFTGDDLRKTDPKFQKPRFEHYLAAVEELKKLAKEHYNKSVLALAIRWMLEQGPTLALWGARKPEQIDGIDEVFGWQISDEDLKQIDAILAKNIPNPIGAEFMAPPPRDK

SEQ ID NO: 3 - DNA, 305 bp, Zymomonas mobilis
gttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctacttaataagttaggagaataaac

SEQ ID NO: 4 - DNA, 304 bp, artificial sequence
  mutated promoter
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaac

SEQ ID NO: 5 - DNA, 304 bp, artificial sequence
  mutant promoter
gttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatataatagctactaataagttaggagaataaac

SEQ ID NO: 6 - DNA, 304 bp, artificial sequence
  double mutation promoter
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatataatagctactaataagttaggagaataaac

SEQ ID NO: 7 - DNA, 305 bp, artificial sequence
  promoter with mutation
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctacttaataagttaggagaataaac

SEQ ID NO: 8 - DNA, 305 bp, artificial sequence
  promoter with mutation
gttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatataatagctacttaataagttaggagaataaac

SEQ ID NO: 9 - DNA, 305 bp, artificial sequence
  promoter with mutations
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatataatagctacttaataagttaggagaataaac

SEQ ID NO: 10 - DNA, 305 bp, artificial sequence
  promoter with mutation
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaac

SEQ ID NO: 11 - DNA, 305 bp, artificial sequence
  promoter with mutation
gttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatatagtagctacttaataagttaggagaataaac

SEQ ID NO: 12 - DNA, 305 bp, artificial sequence
  promoter with mutations
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaataaggtgttggccgcgatcgccggtaagtcggcacgttaaaaaatagctatggaatatagtagctacttaataagttaggagaataaac

SEQ ID NO: 13 - DNA, 1182 bp, artificial sequence
  codon optimized coding region of Actinoplanes missouriensis xylose isomerase for expression in Zymomonas
atgagtgtccaagctacacgcgaagataaatttagctttggcttgtggacagttgggtggcaggcccgtgacgcctttggtgatgccacgcgtaccgcccttgatccggtcgaagcagttcataaacttgccgaaatcggagcctatggcattacatttcatgatgatgacttggtccccttcggttcggatgctcagacacgggatggtatcattgctggtttcaaaaaagcccttgatgaaaccggtttgatcgttcctatggttaccacaaatttgtttacgcatccggtttttaaagacggcgggtttacctctaacgatcgttctgtgcgccggtatgctatccggaaagttttgagacagatggatcttggtgcggaactgggggccaaaacgcttgtcttatggggcggcagagaaggtgcggaatatgattcagcaaaagatgttagtgccgccttggatcgttatcgcgaagcactgaatcttctggcacaatatagcgaagacagaggctatggacttcgttttgcaattgaaccgaaacctaatgaaccacgtggcgatattctgctgcctaccgcaggccatgctattgcctttgtgcaagaattagaacggcccgaactttttggcatcaatccagaaaccgggcatgaacagatgtcaaacttgaattttacccaggggattgctcaggctttgtggcataaaaaactttttcatattgatttgaatggacaacatggtcccaagtttgatcaggaccttgtctttggtcatggtgaccttttaaatgcctttagcctggtcgatttgttagaaaatggtccagatggtgccccggcttatgatggcccgcgccattttgattataaaccatctcgtactgaagattatgatggcgtttgggaatcagcgaaagccaatatccgtatgtatcttttattaaaagaacgtgccaaagcgtttagagctgatccggaagttcaggaagcactggcagcaagcaaagttgccgaattgaaaaccccaacgttgaatcctggtgaaggctatgcagaactgttagcagatcgcagtgcttttgaagattatgatgccgatgctgttggtgcgaaaggttttggtttcgtgaaattgaaccaattggccattgaacatttattaggtgcccgc

SEQ ID NO: 14 - DNA, 945 bp, Zymomonas mobilis
atgaacacttctacgcaaaaacccgctcatttcgacaagatttcgatcaaagggattgataaatccgcaacccgtgtagcgttaggcacatgggctattggtggctggatgtggggcggcactgatgacgatgcctccattaaaaccattcatcgggcgattgatcttggtatcaatatcatcgacaccgctccggcttatggccgtggccatgctgaagaagtcgttggtaaagccatcaaaggtcaacgcgataatttgattattgcgaccaaagtcggccttgattggactttaacccccgaccaatcgatgcgccgtaacagttcagccagccgtatcaaaaaagaaatcgaagattctctgcgccgccttggcactgattatatcgacctttatcaggtgcattggccggatccgctggttccgattgaagaaaccgcaacaatattggaagccctcagaaaagaaggcaaaatccgttctatcggcgtttccaattattccgttcagcagatggacgagttcaagaaatatgccgagctgctggtcgttttaggctatggtgcgctttgccgtggtttactttctggcagaatgacggcggatcgtgcctttacaggcgatgatttacggaaaacagacccgaaattccagaaaccgcgctttgaacattatctggccgcggttgaagaactgaagaaactcgccaaagagcattacaataaatcggtgttggctttggctatccgctggatgttggagcaagggcccactttagcactttggggcgctcgcaagccggaacagatcgacggtattgatgaagtttttggctggcagatatcggatgaagatctgaaacagattgatgctattctggccaagaatatccccaatcctatcggtgcagaatttatggcacccccgccacgcgataaataa

SEQ ID NO: 15 - DNA, 10250 bp, artificial sequence
  constructed plasmid
ctagtgttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaacatgagtgtccaagctacacgcgaagataaatttagctttggcttgtggacagttgggtggcaggcccgtgacgcctttggtgatgccacgcgtaccgcccttgatccggtcgaagcagttcataaacttgccgaaatcggagcctatggcattacatttcatgatgatgacttggtccccttcggttcggatgctcagacacgggatggtatcattgctggtttcaaaaaagcccttgatgaaaccggtttgatcgttcctatggttaccacaaatttgtttacgcatccggtttttaaagacggcgggtttacctctaacgatcgttctgtgcgccggtatgctatccggaaagttttgagacagatggatcttggtgcggaactgggggccaaaacgcttgtcttatggggcggcagagaaggtgcggaatatgattcagcaaaagatgttagtgccgccttggatcgttatcgcgaagcactgaatcttctggcacaatatagcgaagacagaggctatggacttcgttttgcaattgaaccgaaacctaatgaaccacgtggcgatattctgctgcctaccgcaggccatgctattgcctttgtgcaagaattagaacggcccgaactttttggcatcaatccagaaaccgggcatgaacagatgtcaaacttgaattttacccaggggattgctcaggctttgtggcataaaaaactttttcatattgatttgaatggacaacatggtcccaagtttgatcaggaccttgtctttggtcatggtgaccttttaaatgcctttagcctggtcgatttgttagaaaatggtccagatggtgccccggcttatgatggcccgcgccattttgattataaaccatctcgtactgaagattatgatggcgtttgggaatcagcgaaagccaatatccgtatgtatcttttattaaaagaacgtgccaaagcgtttagagctgatccggaagttcaggaagcactggcagcaagcaaagttgccgaattgaaaaccccaacgttgaatcctggtgaaggctatgcagaactgttagcagatcgcagtgcttttgaagattatgatgccgatgctgttggtgcgaaaggttttggtttcgtgaaattgaaccaattggccattgaacatttattaggtgcccgctagtctagatgactgtataaaaccacagccaatcaaacgaaaccaggctatactcaagcctggttttttgatggattttcagcgtggcgcaggcaggttttatcttaacccgacactggcgggacaccccgcaagggacagaagtctccttctggctggcgacggacaacgggccaagcttccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactggtctaaaagattttaagaaaggtttcgatatgtatatcgggatagatcttggcacctcgggcgtaaaagttattttgctcaacgagcagggtgaggtggttgctgcgcaaacggaaaagctgaccgtttcgcgcccgcatccactctggtcggaacaagacccggaacagtggtggcaggcaactgatcgcgcaatgaaagctctgggcgatcagcattctctgcaggacgttaaagcattgggtattgccggccagatgcacggagcaaccttgctggatgctcagcaacgggtgttacgccctgccattttgtggaacgacgggcgctgtgcgcaagagtgcactttgctggaagcgcgagttccgcaatcgcgggtgattaccggcaacctgatgatgcccggatttactgcgcctaaattgctatgggttcagcggcatgagccggagatattccgtcaaatcgacaaagtattattaccgaaagattacttgcgtctgcgtatgacgggggagtttgccagcgatatgtctgacgcagctggcaccatgtggctggatgtcgcaaagcgtgactggagtgacgtcatgctgcaggcttgcgacttatctcgtgaccagatgcccgcattatacgaaggcagcgaaattactggtgctttgttacctgaagttgcgaaagcgtggggtatggcgacggtgccagttgtcgcaggcggtggcgacaatgcagctggtgcagttggtgtgggaatggttgatgctaatcaggcaatgttatcgctggggacgtcgggggtctattttgctgtcagcgaagggttcttaagcaagccagaaagcgccgtacatagcttttgccatgcgctaccgcaacgttggcatttaatgtctgtgatgctgagtgcagcgtcgtgtctggattgggccgcgaaattaaccggcctgagcaatgtcccagctttaatcgctgcagctcaacaggctgatgaaagtgccgagccagtttggtttctgccttatctttccggcgagcgtacgccacacaataatccccaggcgaagggggttttctttggtttgactcatcaacatggccccaatgaactggcgcgagcagtgctggaaggcgtgggttatgcgctggcagatggcatggatgtcgtgcatgcctgcggtattaaaccgcaaagtgttacgttgattgggggcggggcgcgtagtgagtactggcgtcagatgctggcggatatcagcggtcagcagctcgattaccgtacggggggggatgtggggccagcactgggcgcagcaaggctggcgcagatcgcggcgaatccagagaaatcgctcattgaattgttgccgcaactaccgttagaacagtcgcatctaccagatgcgcagcgttatgccgcttatcagccacgacgagaaacgttccgtcgcctctatcagcaacttctgccattaatggcgtaaacgttatcccctgcctgaccgggtgggggataattcacatctatatatctcagtaattaattaatatttagtacgaatttattctgaaaatcatttgttaatggcatttttcagttttgtctttcgttggttactcgtaatgtatcgctggtagatatggagatcgttatgaaaacctcaaagactgtggcaaaactattatttgttgtcggggcgctggtttatctggttgggctatggatctcatgcccattgttaagtggaaaaggctattttcttggcgtgttaatgacagcaacttttggcaactatgcgaattcgcgatcgcataacttcgtataatgtatgctatacgaagttatgcggccgcagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccttaattaaataacttcgtataatgtatgctatacgaagttatggccggcccgttacttgctgggtgaagaaccgatcgaagtccgtgcttacacctacagcgatccgaatgatgaacgtttcgttgaagtcgaagatcgtattatttggcagatgcgcttcagaagcggtgctctgtctcatggtgcatcttcttattcgaccacgacgacttcacgtttctcggtgcagggcgacaaagctgttctgttgatggatccggctaccggatattatcagaatttgatttctgtccagaccccaggccatgctaaccagtcgatgatgccacagttcatcatgccagcgaacaaccagttctctgcacagttggatcatctggctgaagccgtcatcaataacaaaccagttcgtagcccgggtgaagaaggtatgcaggatgtgcgcctgattcaggccatttatgaagcagctcgtaccggtcgccccgtcaacacggattggggttatgtccgtcagggtggttattgattctgacttaacctatttgggttaaacagacttatttttcctgttttaggaaaatagttaaaaaggcgtcattggttcttccaatgacgcctttttttataaacaaaaaaatccttttgtcggttttataaaaatacttcatattttgataagccgtcttaaaaatataataaatttttataatatttatccgatcaaaggacccctttatgctagaagtcattatatcggcattactaccgattataattactttaatgataggttttttcgctggctggcgtggtgaatttacggcaaatcaagcctcgaccttgaataaaatggtcttacgctatgccttacctatgactttattctctgggattttatcacttcccaaaacacagattttatcgtcgggttctgccgcaattattttacttttagccatggctggcggctatctaattacacttgggataggatattttgtctgccagcgcccagtgaatgaatctgctcttttagctctttctgttagcgcacctgcagttccttttgttggcataacagttctagggcatttatttggcactgccagcacgatattggtttcaatatgtagcctgatgatgaacctcgtccaggttcccgttacctttttcttttgtcagcgtattctccaaaaaagaatactgacaaaatagccacggatagttcttttttttctcatatcagacatgcttttaccgaacctattgttattgcccctattctggctcttatctgtgtcagtctctctattcctttccctgaaaccttaaaatcttctttaatgctactaggaaaagcgaccggaggcgttgcgcttttctcttctggtataattttattttctcgaaaagttattttaagtaaaacagtagcatctttagttttatcaaaaaatattattattccaacagcggtattggttcttgcgtcggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctcgtccagaaaagacagcattccttctcaataaagaaatattattttttgtttttgaaaaatttttccaaaatctagaatgctacattaaatatacaaaaatattattatacaaataaggcttttaaatacccatattttttagaatttctttacaaagaaacatgttaaatatagatttagagattaatatcagccatttttatcaaaaattctttttttgttttataatattatgctgcaaaactaataaaaacgccctttcgaaattaacgatcacccacaagaaataattatctgacagcgcttaccaatcaattattgccgaacgcagagtcccgtattaggacggtcaacaatctaaaccgtttttcagaaaatattgctttataagcctcaaaacttaaaagctgcggtattttaatataccaaaattttctggaaaagccggcgaatcagataacagttccgcacaggtgagaaccacgacggatcttctctgaattgttggttagttaagaaagaaacaaggattatgacgaacaaaatctcgtcttcagataatctttccaatgctgtttcagcaacggatgacaacgcttcccgtacgccaaatctgacccgtcgcgctctcgttggtggtggtgttggactggccgcagctggcgccttagccagtggtcttcaggcagcgacgcttcctgctggtgccagccaggttccgaccacgcctgcaggtcgcccgatgccttacgcgatccgcccgatgccggaagatcgtcgtttcggttatgctatcgtcggtctgggtaaatatgcccttaaccagattttaccgggttttgccggatgccagcattcccgcatcgaagctttggtcagcggtaacgctgaaaaagctaaaatcgttgccgctgaatatggcgtcgatccccgtaaaatttatgattacagcaacttcgacaagatcgctaaagatccaaaaatcgacgctgtttacatcattttgccaaactctttgcatgctgaatttgctatccgtgctttcaaagccggcaagcatgttatgtgtgaaaagccgatggcaacctctgttgctgattgtcagcggatgatcgatgcagccaaggctgctaataaaaagctgatgatcggttaccgttgccactatgatccaatgaaccgtgcaa

SEQ ID NO: 16 - DNA, 1186 bp, artificial sequence
  sonstructed fragment
gtccagaaaagacagcattccttctcaataaagaaatattattttttgtttttgaaaaatttttccaaaatctagaatgctacattaaatatacaaaaatattattatacaaataaggcttttaaatacccatattttttagaatttctttacaaagaaacatgttaaatatagatttagagattaatatcagccatttttatcaaaaattctttttttgttttataatattatgctgcaaaactaataaaaacgccctttcgaaattaacgatcacccacaagaaataattatctgacagcgcttaccaatcaattattgccgaacgcagagtcccgtattaggacggtcaacaatctaaaccgtttttcagaaaatattgctttataagcctcaaaacttaaaagctgcggtattttaatataccaaaattttctggaaaagccggcgaatcagataacagttccgcacaggtgagaaccacgacggatcttctctgaattgttggttagttaagaaagaaacaaggattatgacgaacaaaatctcgtcttcagataatctttccaatgctgtttcagcaacggatgacaacgcttcccgtacgccaaatctgacccgtcgcgctctcgttggtggtggtgttggactggccgcagctggcgccttagccagtggtcttcaggcagcgacgcttcctgctggtgccagccaggttccgaccacgcctgcaggtcgcccgatgccttacgcgatccgcccgatgccggaagatcgtcgtttcggttatgctatcgtcggtctgggtaaatatgcccttaaccagattttaccgggttttgccggatgccagcattcccgcatcgaagctttggtcagcggtaacgctgaaaaagctaaaatcgttgccgctgaatatggcgtcgatccccgtaaaatttatgattacagcaacttcgacaagatcgctaaagatccaaaaatcgacgctgtttacatcattttgccaaactctttgcatgctgaatttgctatccgtgctttcaaagccggcaagcatgttatgtgtgaaaagccgatggcaacctctgttgctgattgtcagcggatgatcgatgcagccaaggctgctaataaaaagctgatgatcggttaccgttgccactatgatccaatgaaccgtgca

SEQ ID NO: 17 - DNA, 1302 bp, Zymomonas mobilis
atgacgaacaaaatctcgtcttcagataatctttccaatgctgtttcagcaacggatgacaacgcttcccgtacgccaaatctgacccgtcgcgctctcgttggtggtggtgttggactggccgcagctggcgccttagccagtggtcttcaggcagcgacgcttcctgctggtgccagccaggttccgaccacgcctgcaggtcgcccgatgccttacgcgatccgcccgatgccggaagatcgtcgtttcggttatgctatcgtcggtctgggtaaatatgcccttaaccagattttaccgggttttgccggatgccagcattcccgcatcgaagctttggtcagcggtaacgctgaaaaagctaaaatcgttgccgctgaatatggcgtcgatccccgtaaaatttatgattacagcaacttcgacaagatcgctaaagatccaaaaatcgacgctgtttacatcattttgccaaactctttgcatgctgaatttgctatccgtgctttcaaagccggcaagcatgttatgtgtgaaaagccgatggcaacctctgttgctgattgtcagcggatgatcgatgcagccaaggctgctaataaaaagctgatgatcggttaccgttgccactatgatccaatgaaccgtgcagcggtaaaattgatccgtgaaaaccagttgggtaaactgggcatggttaccaccgacaactcagacgttatggatcagaacgatcctgcacagcagtggcgtctgcgtcgtgaactcgccggtggcggttctttgatggatatcggtatttatggcttgaacggtacccgttacttgctgggtgaagaaccgatcgaagtccgtgcttacacctacagcgatccgaatgatgaacgtttcgttgaagtcgaagatcgtattatttggcagatgcgcttcagaagcggtgctctgtctcatggtgcatcttcttattcgaccacgacgacttcacgtttctcggtgcagggcgacaaagctgttctgttgatggatccggctaccggatattatcagaatttgatttctgtccagaccccaggccatgctaaccagtcgatgatgccacagttcatcatgccagcgaacaaccagttctctgcacagttggatcatctggctgaagccgtcatcaataacaaaccagttcgtagcccgggtgaagaaggtatgcaggatgtgcgcctgattcaggccatttatgaagcagctcgtaccggtcgccccgtcaacacggattggggttatgtccgtcagggtggttattga

SEQ ID NO: 18 - DNA, 1446 bp, artificial sequence
  constructed fragment
cgttacttgctgggtgaagaaccgatcgaagtccgtgcttacacctacagcgatccgaatgatgaacgtttcgttgaagtcgaagatcgtattatttggcagatgcgcttcagaagcggtgctctgtctcatggtgcatcttcttattcgaccacgacgacttcacgtttctcggtgcagggcgacaaagctgttctgttgatggatccggctaccggatattatcagaatttgatttctgtccagaccccaggccatgctaaccagtcgatgatgccacagttcatcatgccagcgaacaaccagttctctgcacagttggatcatctggctgaagccgtcatcaataacaaaccagttcgtagcccgggtgaagaaggtatgcaggatgtgcgcctgattcaggccatttatgaagcagctcgtaccggtcgccccgtcaacacggattggggttatgtccgtcagggtggttattgattctgacttaacctatttgggttaaacagacttatttttcctgttttaggaaaatagttaaaaaggcgtcattggttcttccaatgacgcctttttttataaacaaaaaaatccttttgtcggttttataaaaatacttcatattttgataagccgtcttaaaaatataataaatttttataatatttatccgatcaaaggacccctttatgctagaagtcattatatcggcattactaccgattataattactttaatgataggttttttcgctggctggcgtggtgaatttacggcaaatcaagcctcgaccttgaataaaatggtcttacgctatgccttacctatgactttattctctgggattttatcacttcccaaaacacagattttatcgtcgggttctgccgcaattattttacttttagccatggctggcggctatctaattacacttgggataggatattttgtctgccagcgcccagtgaatgaatctgctcttttagctctttctgttagcgcacctgcagttccttttgttggcataacagttctagggcatttatttggcactgccagcacgatattggtttcaatatgtagcctgatgatgaacctcgtccaggttcccgttacctttttcttttgtcagcgtattctccaaaaaagaatactgacaaaatagccacggatagttcttttttttctcatatcagacatgcttttaccgaacctattgttattgcccctattctggctcttatctgtgtcagtctctctattcctttccctgaaaccttaaaatcttctttaatgctactaggaaaagcgaccggaggcgttgcgcttttctcttctggtataattttattttctcgaaaagttattttaagtaaaacagtagcatctttagttttatcaaaaaatattattattccaacagcggtattggttcttgcgtc

SEQ ID NO: 19 - DNA, 1661 bp, artificial sequence
  constructed fragment
gttcgatcaacaacccgaatcctatcgtaatgatgttttgcccgatcagcctcaatcgacaattttacgcgtttcgatcgaagcagggacgacaattggctgggaacggtatacttgaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaacatgagtgtccaagctacacgcgaagataaatttagctttggcttgtggacagttgggtggcaggcccgtgacgcctttggtgatgccacgcgtaccgcccttgatccggtcgaagcagttcataaacttgccgaaatcggagcctatggcattacatttcatgatgatgacttggtccccttcggttcggatgctcagacacgggatggtatcattgctggtttcaaaaaagcccttgatgaaaccggtttgatcgttcctatggttaccacaaatttgtttacgcatccggtttttaaagacggcgggtttacctctaacgatcgttctgtgcgccggtatgctatccggaaagttttgagacagatggatcttggtgcggaactgggggccaaaacgcttgtcttatggggcggcagagaaggtgcggaatatgattcagcaaaagatgttagtgccgccttggatcgttatcgcgaagcactgaatcttctggcacaatatagcgaagacagaggctatggacttcgttttgcaattgaaccgaaacctaatgaaccacgtggcgatattctgctgcctaccgcaggccatgctattgcctttgtgcaagaattagaacggcccgaactttttggcatcaatccagaaaccgggcatgaacagatgtcaaacttgaattttacccaggggattgctcaggctttgtggcataaaaaactttttcatattgatttgaatggacaacatggtcccaagtttgatcaggaccttgtctttggtcatggtgaccttttaaatgcctttagcctggtcgatttgttagaaaatggtccagatggtgccccggcttatgatggcccgcgccattttgattataaaccatctcgtactgaagattatgatggcgtttgggaatcagcgaaagccaatatccgtatgtatcttttattaaaagaacgtgccaaagcgtttagagctgatccggaagttcaggaagcactggcagcaagcaaagttgccgaattgaaaaccccaacgttgaatcctggtgaaggctatgcagaactgttagcagatcgcagtgcttttgaagattatgatgccgatgctgttggtgcgaaaggttttggtttcgtgaaattgaaccaattggccattgaacatttattaggtgcccgctagtctagatgactgtataaaaccacagccaatcaaacgaaaccaggctatactcaagcctggttttttgatggattttcagcgtggcgcaggcaggttttatcttaacccgacactggcgggacaccccgcaagggacagaagtctccttctggctggcgacggacaacgggcc

SEQ ID NO: 20 - DNA, 1960 bp, artificial sequence
  constructed fragment containing a 191 bp Peno, a 1,455-bp E. coli xylB coding sequence (ECxylB) and a 314-bp E.coli xylB 3′UTR
ccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactggtctaaaagattttaagaaaggtttcgatatgtatatcgggatagatcttggcacctcgggcgtaaaagttattttgctcaacgagcagggtgaggtggttgctgcgcaaacggaaaagctgaccgtttcgcgcccgcatccactctggtcggaacaagacccggaacagtggtggcaggcaactgatcgcgcaatgaaagctctgggcgatcagcattctctgcaggacgttaaagcattgggtattgccggccagatgcacggagcaaccttgctggatgctcagcaacgggtgttacgccctgccattttgtggaacgacgggcgctgtgcgcaagagtgcactttgctggaagcgcgagttccgcaatcgcgggtgattaccggcaacctgatgatgcccggatttactgcgcctaaattgctatgggttcagcggcatgagccggagatattccgtcaaatcgacaaagtattattaccgaaagattacttgcgtctgcgtatgacgggggagtttgccagcgatatgtctgacgcagctggcaccatgtggctggatgtcgcaaagcgtgactggagtgacgtcatgctgcaggcttgcgacttatctcgtgaccagatgcccgcattatacgaaggcagcgaaattactggtgctttgttacctgaagttgcgaaagcgtggggtatggcgacggtgccagttgtcgcaggcggtggcgacaatgcagctggtgcagttggtgtgggaatggttgatgctaatcaggcaatgttatcgctggggacgtcgggggtctattttgctgtcagcgaagggttcttaagcaagccagaaagcgccgtacatagcttttgccatgcgctaccgcaacgttggcatttaatgtctgtgatgctgagtgcagcgtcgtgtctggattgggccgcgaaattaaccggcctgagcaatgtcccagctttaatcgctgcagctcaacaggctgatgaaagtgccgagccagtttggtttctgccttatctttccggcgagcgtacgccacacaataatccccaggcgaagggggttttctttggtttgactcatcaacatggccccaatgaactggcgcgagcagtgctggaaggcgtgggttatgcgctggcagatggcatggatgtcgtgcatgcctgcggtattaaaccgcaaagtgttacgttgattgggggcggggcgcgtagtgagtactggcgtcagatgctggcggatatcagcggtcagcagctcgattaccgtacggggggggatgtggggccagcactgggcgcagcaaggctggcgcagatcgcggcgaatccagagaaatcgctcattgaattgttgccgcaactaccgttagaacagtcgcatctaccagatgcgcagcgttatgccgcttatcagccacgacgagaaacgttccgtcgcctctatcagcaacttctgccattaatggcgtaaacgttatcccctgcctgaccgggtgggggataattcacatctatatatctcagtaattaattaatatttagtacgaatttattctgaaaatcatttgttaatggcatttttcagttttgtctttcgttggttactcgtaatgtatcgctggtagatatggagatcgttatgaaaacctcaaagactgtggcaaaactattatttgttgtcggggcgctggtttatctggttgggctatggatctcatgcccattgttaagtggaaaaggctattttcttggcgtgttaatgacagcaacttttggcaactatgc

SEQ ID NO: 21 - DNA, 1014 bp, artificial sequence
  constructed fragment with spec resistance gene bounded by lox recombination sites
agcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgactgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgcc

SEQ ID NO: 22 - DNA, 12198 bp, artificial sequence
  constructed plasmid
ctagtgttcgatcaacaacccgaatcctatcgtaatgatgttttgcccgatcagcctcaatcgacaattttacgcgtttcgatcgaaacagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaacatgacggacaaattgacctcccttcgtcagtacaccaccgtagtggccgacactggggacatcgcggcaatgaagctgtatcaaccgcaggatgccacaaccaacccttctctcattcttaacgcagcgcagattccggaataccgtaagttgattgatgatgctgtcgcctgggcgaaacagcagagcaacgatcgcgcgcagcagatcgtggacgcgaccgacaaactggcagtaaatattggtctggaaatcctgaaactggttccgggccgtatctcaactgaagttgatgcgcgcctttcctatgacaccgaagcgtcaattgcgaaagcaaaacgcctgatcaaactctacaacgatgctggtattagcaacgatcgtattctgatcaaactggcttctacctggcagggtatccgtgctgcagaacagctggaaaaagaaggcatcaactgtaacctgaccctgctgttctccttcgctcaggctcgtgcttgtgcggaagcgggcgtgttcctgatctcgccgtttgttggccgtattcttgactggtacaaagcgaataccgataagaaagagtacgctccggcagaagatccgggcgtggtttctgtatctgaaatctaccagtactacaaagagcacggttatgaaaccgtggttatgggcgcaagcttccgtaacatcggcgaaattctggaactggcaggctgcgaccgtctgaccatcgcaccggcactgctgaaagagctggcggagagcgaaggggctatcgaacgtaaactgtcttacaccggcgaagtgaaagcgcgtccggcgcgtatcactgagtccgagttcctgtggcagcacaaccagggtccaatggcagtagataaactggcggaaggtatccgtaagtttgctattgaccaggaaaaactggaaaaaatgatcggcgatctgctgtaatctagacgatctggagtcaaaatgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaattagcatttcgggtaaaaaaggtcgcttcggcgaccttttttattaccttgataatgtccgtttgcgcggcgcgccccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactggtctaaaagattttaagaaaggtttcgatatgacctctgctgtgccatcaaatacgaaaaaaaagctggtgattgcttccgatcacgcagcatttgagttgaaatcaaccttgattacttggctgaaagagcttggtcatgaggtcgaagaccttggccctcatgaaaaccattcagtcgattatcccgattacggttataagctggctgtcgctatcgcagaaaaaaccgctgatttcggtattgctttatgtggctcgggaatcggtatctcgatcgctgtcaatcgccatccggctgcccgttgcgctttgattacggataaccttaccgcccgtttggcaagagaacataacaatgccaatgttatcgctatgggtgcgagattgatcggcattgaaaccgctaaggattgtatttcagctttccttgcaacgccgtttggaggtgaacgtcatgttcgccgtatcgataaactttcgaatcctcagttcaatatctagataagttaggagaataaacatgagtaaattacccctgattgctccctctatcctttcggcggattttgcccatttgggagatgaggtcgcggcgatagatcaggccggtgccgattggatccatattgatgtgatggatggccatttcgtgccgaatatcaccataggccccatggttgtgaaggctttgcgtccctatagccaaaagccttttgatgtccatttgatgattgcgcctgtcgatcaatatatcgaggctttttctgaagcgggtgctgatattatcagtttccatcccgaagcgggcgcgcatccccatcgcactattcagcatatcaaatcattgggcaaaaaagcgggattagtttttaatccggcgacccctttaagctggcttgattatctaatggatgatcttgatctgattatggtgatgagcgttaaccccggttttggcggccaaaaatttatcaaaacccaattagaaaagattaaagatatccgtcaaagaattaccgcctctgggcgggatatccgcttggaagtggatggcggaattgatgccacgactgcaccgcttgccgtcgaagccggtgccgatgttttggtcgcgggaacggccagctttaaaggcggcgcaacatgttacaccgataatatcaggatattgcgtaaatcatgattaattaactcgaggcggcctgaacgtactgcaagtcctgacgtcactgtgcagtccgttggcccggttatcggtagcgataccgggcatttttttaaggaacgatcgatagaattcgcgggccggccaagcttgaattcatggttttggtgccaatgttatcgcctataaaccgcatccagaccccgaattggcgaaaaaggtcggtttccgcttcacctctctcgatgaagtgatcgagaccagcgacatcatttcgcttcactgtccgctcacgccagaaaatcatcacatgattaatgaagaaacactggcaagggcaaaaaaaggcttttacctcgtcaataccagtcgcggcggcttggttgataccaaggcggtgattaaatcgctgaaagccaaacatctcggcggttatgcggcggatgtttacgaagaggaggggcctttattcttcgaaaatcacgctgacgatattatcgaagatgatattctcgaaaggttgatcgctttcccgaatgtggttttcacgggacatcaggcctttttgacgaaagaggccttatcaaacattgctcacagtattctacaagatatcagcgatgccgaagctggaaaagaaatgccggatgcgcttgtttagtagacaagcgacaattaaccttttgaagatcataatgatcaaatttttgggttaattcggtagttatggcataggctattacgcgctaattgatatcaaaaaaaagcatagccggacatcataccggctatgttttttattaggaaaaaatttcctttcaccttgcttagccatcgccgcattatttaatcaatatgccgagtttttcttgaaatccctatcttacaccaaggccaacaagggaatcatccatactcggtgtcctatcctatgactttttaaattttctccaaatttactaaaatcacgccatctcagcggctgctattttcaaaaagcgcctctcaaaaccgctttttcctgctcaaatatcggatcccaaaattccctcaaaaaaggcagggtattttttacaaaatcgcccctaatatctctcaatccgctgccttgttcatatgtttttgcaaatgatttttattaaacttttttaggcgtatttttatcaagaaaatttaaataatcacatttttattattttagatttaagtattgatacaagtgatatctataaatgtttttataactttctggatcgtaatcggctggcaatcgttttccctatattcgcaagatgtatgtcagccgcgcggccgcctaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgatgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtcgacctttgtagtcttggcctgttgtgtgcatgagcaaatcaatggcaccaccccctcctttttgagctgaatggtcataaaatttataattatctatcgtaattcggaatctatgttcagggtctcgccattgctttttgtctgctgggtcaagttccatgcctaaggtttttaagacatcagaaagaggtattgcacgcatgctatcagcttttcttctagctaatgacagggcttcctctgctctatctgctcgttttttttcttccacatatctcgccgctttgtcagccagcggctgtattacggaaagtgccgatttttgggcttttaggcgttctttttctgcccattcttccttatttgtaaaaattgagggtgggatgggtgcctgaatcttgggatctagctgtaaagttttgttgatatttccgtaatgtctttggactctttgatgcgttgcttttgaaccttttacgcctctggccagccctagaggctccatagaagccgcataatccgtctggagggcagaaagggcttttcgaccatcaaaccatctcgatgcgtttaaacggcctgtatcggggtctctaggcaccataaagccggttaagtggggtgttgtttcatcagcatgtagctgaagagatacaaggttgttttctccaaaggtttgttccgcccattgctgggtgattgttttccagtgttcgagtttttcaggagtggcctgttttgaccattctggagacataccaaagaacagttctatggcctgcacaccgttttttctaagaggctttcccgtttctttctgaattttattcagcatagatttaacatctgctgatgggtcagtagagcctttgagtatttcgtttagttcttttctatctgggtcagcgttttgtgtttcgcggcctcgcgtcatatgcaggctcgcggctttaatcgtgccaactgttttatgtttttcaaacctaaagattgcatagttcggcatgttttaactgctttaatttgagaaaagaccagaggaaataatccagcctatatttctttccctagtagcgaactggaattgtttttccgaaggaaaaaagcaattccgtagtgagtactgaatttattctgattcgtcttgcttttggagcgtctttttgcgttctataactgttgtgaaagctacgcggtcgccattgaaaacgaaattaggattaataaaataccatccttggcgaacatgctttgcaatgattttagctttttctaattcggctagacctcttgcaaaggtagcttgagatagtgccagttttttttcttgtgcgttaagaaagtcctctaaaacgaatttgtctaaagggacgaggtctttgctgatgcctttgtcttgaagtatccaaaccagaacgctgaaagcttttattccagcggctcctagttcaaaagttagcgcgatattggtgctaaataattttacaaattcttcactatcaacacgtctgtaagtcgtcacatgagtgccttgcatctcaccagtggcttgattgaccagaatgttatcatctcgtcctaatcgagataactgaaccctctgacttttaactggcacaaccataccttcgatgaaaggattctcgtcatatctgattggctgctttctcaattttgtcgccatatttgataaacctttaatcaaaaaaaccacattttttgattatacctattcatcgaatgaggcaaggtctatcaattttacccctttttttgatagacggtttaatcaatattgatagaccccttcacagattctgaaaatcgacttccctattttagggatattttcacgattccctttcttagttcttcctagtggggaaattcgttgaatcctgcctcggaaaaaccatgagaaagctgttggttatatacacgggcaaagccaccctatttttagctactggggaaagagataaggcagggtatttgtaaaattaaaaccggatttttcgctttacggtttgtttaggcgcaactgtctttttaagaccgcgtttaaccatcaaaagatcgttccaatcttttccgtgtatcatctgttctttaggtgggagccagttttcaactttttttgttggaaacgcggctttaatcgctccgactaatagcgatgctgctctttgtcctacagcatcccaatcataggcaatatggacagaagatgccttttcaacgatttttcggagagttttagtaagagacgttcttacgccgctggtgcttaataattttacgccagctttaattttttctgggcttaaaaagccgactactgaaatcgcgtctatcgcactttcagcgatataaagatcatacttttcgtcattttttacattgatgctgccagtaaaatgggcttcgcgactgcttcccaaggctaaccctttaaaaccactgcttgttccgcgtaattctgcgccctgaagtgtatctttatcgtcatacatcaagaaggctacattaccgcgatcatctgttcggatagagtcaggaatattgttaaatgatattcctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgggagctcgtttttctatccccatcacctcggttttgttgacaaaaaaaggtggccactaaattggctttccgcaccgatgggatgatttttattctttgctattcttcgctctttgcccaattcattaaaagcggaaatcatcaccaaagatagaagacgcagccttcaccatttcagattgcccttctcgggcattttctgctgctagaatcctcttaaaaatattaaattccactctattggtaatatgtttccctctttagggaacaaataaagcccttctttgttctataaaagttagcttaccgattttacaaaaaataataccgcttcattcaatcggtaatacatatcttttttcttcaaaaaacttttcaagagggtgtctatgcgcgtcgcaatattcagttccaaaaactatgaccatcattctattgaaaaagaaaatgaacattatggccatgaccttgtttttctgaatgagcggcttaccaaagagacagcagaaaaagccaaagacgcagaagctgtttgtatctttgtgaatgacgaagccaatgccgaagtgctggaaattttggcaggcttaggcatcaagttggttgctcttcgttgcgccggttataacaatgtcgatctcgatgcggccaaaaagctgaatatcaaggttgtgcgcgtgcctgcctattcgccctattcggttgccgaatatgcagtagggatgttgctcaccctgaatcggcaaatttcacgcggtttgaagcgggttcgggaaaataacttctccttggaaggtttgattggccttgatgtgcatgacaaaacagtcggcattatcggtgttggtcatatcgggagcgtctttgcccatattatgaccca

SEQ ID NO: 23 - DNA, 875 bp, artificial sequence
  constructed fragment for targeting integration into the ldh locus of Z. mobilis
gtttttctatccccatcacctcggttttgttgacaaaaaaaggtggccactaaattggctttccgcaccgatgggatgatttttattctttgctattcttcgctctttgcccaattcattaaaagcggaaatcatcaccaaagatagaagacgcagccttcaccatttcagattgcccttctcgggcattttctgctgctagaatcctcttaaaaatattaaattccactctattggtaatatgtttccctctttagggaacaaataaagcccttctttgttctataaaagttagcttaccgattttacaaaaaataataccgcttcattcaatcggtaatacatatcttttttcttcaaaaaacttttcaagagggtgtctatgcgcgtcgcaatattcagttccaaaaactatgaccatcattctattgaaaaagaaaatgaacattatggccatgaccttgtttttctgaatgagcggcttaccaaagagacagcagaaaaagccaaagacgcagaagctgtttgtatctttgtgaatgacgaagccaatgccgaagtgctggaaattttggcaggcttaggcatcaagttggttgctcttcgttgcgccggttataacaatgtcgatctcgatgcggccaaaaagctgaatatcaaggttgtgcgcgtgcctgcctattcgccctattcggttgccgaatatgcagtagggatgttgctcaccctgaatcggcaaatttcacgcggtttgaagcgggttcgggaaaataacttctccttggaaggtttgattggccttgatgtgcatgacaaaacagtcggcattatcggtgttggtcatatcgggagcgtctttgcccatattatgaccc

SEQ ID NO: 24 - DNA, 1149 bp, artificial sequence
  constructed fragment for integration into the Z. mobilis ldh locus
atggttttggtgccaatgttatcgcctataaaccgcatccagaccccgaattggcgaaaaaggtcggtttccgcttcacctctctcgatgaagtgatcgagaccagcgacatcatttcgcttcactgtccgctcacgccagaaaatcatcacatgattaatgaagaaacactggcaagggcaaaaaaaggcttttacctcgtcaataccagtcgcggcggcttggttgataccaaggcggtgattaaatcgctgaaagccaaacatctcggcggttatgcggcggatgtttacgaagaggaggggcctttattcttcgaaaatcacgctgacgatattatcgaagatgatattctcgaaaggttgatcgctttcccgaatgtggttttcacgggacatcaggcctttttgacgaaagaggccttatcaaacattgctcacagtattctacaagatatcagcgatgccgaagctggaaaagaaatgccggatgcgcttgtttagtagacaagcgacaattaaccttttgaagatcataatgatcaaatttttgggttaattcggtagttatggcataggctattacgcgctaattgatatcaaaaaaaagcatagccggacatcataccggctatgttttttattaggaaaaaatttcctttcaccttgcttagccatcgccgcattatttaatcaatatgccgagtttttcttgaaatccctatcttacaccaaggccaacaagggaatcatccatactcggtgtcctatcctatgactttttaaattttctccaaatttactaaaatcacgccatctcagcggctgctattttcaaaaagcgcctctcaaaaccgctttttcctgctcaaatatcggatcccaaaattccctcaaaaaaggcagggtattttttacaaaatcgcccctaatatctctcaatccgctgccttgttcatatgtttttgcaaatgatttttattaaacttttttaggcgtatttttatcaagaaaatttaaataatcacatttttattattttagatttaagtattgatacaagtgatatctataaatgtttttataactttctggatcgtaatcggctggcaatcgttttccctatattcgcaagatgtatgtcagccgc

SEQ ID NO: 25 - DNA, 996 bp, Zymomonas mobilis
atgcgcgtcgcaatattcagttccaaaaactatgaccatcattctattgaaaaagaaaatgaacattatggccatgaccttgtttttctgaatgagcggcttaccaaagagacagcagaaaaagccaaagacgcagaagctgtttgtatctttgtgaatgacgaagccaatgccgaagtgctggaaattttggcaggcttaggcatcaagttggttgctcttcgttgcgccggttataacaatgtcgatctcgatgcggccaaaaagctgaatatcaaggttgtgcgcgtgcctgcctattcgccctattcggttgccgaatatgcagtagggatgttgctcaccctgaatcggcaaatttcacgcggtttgaagcgggttcgggaaaataacttctccttggaaggtttgattggccttgatgtgcatgacaaaacagtcggcattatcggtgttggtcatatcgggagcgtctttgcccatattatgacccatggttttggtgccaatgttatcgcctataaaccgcatccagaccccgaattggcgaaaaaggtcggtttccgcttcacctctctcgatgaagtgatcgagaccagcgacatcatttcgcttcactgtccgctcacgccagaaaatcatcacatgattaatgaagaaacactggcaagggcaaaaaaaggcttttacctcgtcaataccagtcgcggcggcttggttgataccaaggcggtgattaaatcgctgaaagccaaacatctcggcggttatgcggcggatgtttacgaagaggaggggcctttattcttcgaaaatcacgctgacgatattatcgaagatgatattctcgaaaggttgatcgctttcccgaatgtggttttcacgggacatcaggcctttttgacgaaagaggccttatcaaacattgctcacagtattctacaagatatcagcgatgccgaagctggaaaagaaatgccggatgcgcttgtttag

SEQ ID NO: 26 - DNA, 3339 bp, artificial sequence
  constructed fragment containing a 304-bp T-mutant of the Z. mobilis GAP promoter (PgapT), a 954-bp E. coli Tal coding region (ECTal), a 1,992-bp E. coli Tkt coding region, and a 68-bp E. coli Tkt 3′UTR (ECTkt 3′UTR)
gttcgatcaacaacccgaatcctatcgtaatgatgttttgcccgatcagcctcaatcgacaattttacgcgtttcgatcgaaacagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaacatgacggacaaattgacctcccttcgtcagtacaccaccgtagtggccgacactggggacatcgcggcaatgaagctgtatcaaccgcaggatgccacaaccaacccttctctcattcttaacgcagcgcagattccggaataccgtaagttgattgatgatgctgtcgcctgggcgaaacagcagagcaacgatcgcgcgcagcagatcgtggacgcgaccgacaaactggcagtaaatattggtctggaaatcctgaaactggttccgggccgtatctcaactgaagttgatgcgcgcctttcctatgacaccgaagcgtcaattgcgaaagcaaaacgcctgatcaaactctacaacgatgctggtattagcaacgatcgtattctgatcaaactggcttctacctggcagggtatccgtgctgcagaacagctggaaaaagaaggcatcaactgtaacctgaccctgctgttctccttcgctcaggctcgtgcttgtgcggaagcgggcgtgttcctgatctcgccgtttgttggccgtattcttgactggtacaaagcgaataccgataagaaagagtacgctccggcagaagatccgggcgtggtttctgtatctgaaatctaccagtactacaaagagcacggttatgaaaccgtggttatgggcgcaagcttccgtaacatcggcgaaattctggaactggcaggctgcgaccgtctgaccatcgcaccggcactgctgaaagagctggcggagagcgaaggggctatcgaacgtaaactgtcttacaccggcgaagtgaaagcgcgtccggcgcgtatcactgagtccgagttcctgtggcagcacaaccagggtccaatggcagtagataaactggcggaaggtatccgtaagtttgctattgaccaggaaaaactggaaaaaatgatcggcgatctgctgtaatctagacgatctggagtcaaaatgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaattagcatttcgggtaaaaaaggtcgcttcggcgaccttttttattaccttgataatgtccgtttgcgc

SEQ ID NO: 27 - DNA, 304 bp, artificial sequence
  mutant Pgap promoter with a “G” to an “A” change at position 83 in SEQ ID NO21
gttcgatcaacaacccgaatcctatcgtaatgatgttttgcccgatcagcctcaatcgacaattttacgcgtttcgatcgaaacagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaac

SEQ ID NO: 28 - DNA, 1443 bp, artificial sequence
  constructed fragment containing a 191 bp Peno, a 471 bp Z. mobilis Rpi coding sequence (ZMRpi), a 663 bp Z. mobilis Rpe coding sequence (ZMRpe), and a 35 bp E.coli xylA 3′UTR (ECxylA 3′UTR)
ccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactggtctaaaagattttaagaaaggtttcgatatgacctctgctgtgccatcaaatacgaaaaaaaagctggtgattgcttccgatcacgcagcatttgagttgaaatcaaccttgattacttggctgaaagagcttggtcatgaggtcgaagaccttggccctcatgaaaaccattcagtcgattatcccgattacggttataagctggctgtcgctatcgcagaaaaaaccgctgatttcggtattgctttatgtggctcgggaatcggtatctcgatcgctgtcaatcgccatccggctgcccgttgcgctttgattacggataaccttaccgcccgtttggcaagagaacataacaatgccaatgttatcgctatgggtgcgagattgatcggcattgaaaccgctaaggattgtatttcagctttccttgcaacgccgtttggaggtgaacgtcatgttcgccgtatcgataaactttcgaatcctcagttcaatatctagataagttaggagaataaacatgagtaaattacccctgattgctccctctatcctttcggcggattttgcccatttgggagatgaggtcgcggcgatagatcaggccggtgccgattggatccatattgatgtgatggatggccatttcgtgccgaatatcaccataggccccatggttgtgaaggctttgcgtccctatagccaaaagccttttgatgtccatttgatgattgcgcctgtcgatcaatatatcgaggctttttctgaagcgggtgctgatattatcagtttccatcccgaagcgggcgcgcatccccatcgcactattcagcatatcaaatcattgggcaaaaaagcgggattagtttttaatccggcgacccctttaagctggcttgattatctaatggatgatcttgatctgattatggtgatgagcgttaaccccggttttggcggccaaaaatttatcaaaacccaattagaaaagattaaagatatccgtcaaagaattaccgcctctgggcgggatatccgcttggaagtggatggcggaattgatgccacgactgcaccgcttgccgtcgaagccggtgccgatgttttggtcgcgggaacggccagctttaaaggcggcgcaacatgttacaccgataatatcaggatattgcgtaaatcatgattaattaactcgaggcggcctgaacgtactgcaagtcctgacgtcactgtgcagtccgttggcccggttatcggtagcgataccgggcattttttt

SEQ ID NO: 29 - DNA, 12704 bp, artificial sequence
  constructed plasmid
ctagtgttcgatcaacaacccgaatcctatcgtaatgatgttttgcccgatcagcctcaatcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatataatagctactaataagttaggagaataaacatgacggacaaattgacctcccttcgtcagtacaccaccgtagtggccgacactggggacatcgcggcaatgaagctgtatcaaccgcaggatgccacaaccaacccttctctcattcttaacgcagcgcagattccggaataccgtaagttgattgatgatgctgtcgcctgggcgaaacagcagagcaacgatcgcgcgcagcagatcgtggacgcgaccgacaaactggcagtaaatattggtctggaaatcctgaaactggttccgggccgtatctcaactgaagttgatgcgcgtctttcctatgacaccgaagcgtcaattgcgaaagcaaaacgcctgatcaaactctacaacgatgctggtattagcaacgatcgtattctgatcaaactggcttctacctggcagggtatccgtgctgcagaacagctggaaaaagaaggcatcaactgtaacctgaccctgctgttctccttcgctcaggctcgtgcttgtgcggaagcgggcgtgttcctgatctcgccgtttgttggccgtattcttgactggtacaaagcgaataccgataagaaagagtacgctccggcagaagatccgggcgtggtttctgtatctgaaatctaccagtactacaaagagcacggttatgaaaccgtggttatgggcgcaagcttccgtaacatcggcgaaattctggaactggcaggctgcgaccgtctgaccatcgcaccggcactgctgaaagagctggcggagagcgaaggggctatcgaacgtaaactgtcttacaccggcgaagtgaaagcgcgtccggcgcgtatcactgagtccgagttcctgtggcagcacaaccaggatccaatggcagtagataaactggcggaaggtatccgtaagtttgctattgaccaggaaaaactggaaaaaatgatcggcgatctgctgtaatctagacgatctggagtcaaaatgtcctcacgtaaagagcttgccaatgctattcgtgcgctgagcatggacgcagtacagaaagccaaatccggtcacccgggggcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacgggccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgtcgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaattagcatttcgggtaaaaaaggtcgcttcggcgaccttttttattaccttgataatgtccgtttgcgcggcgcgccccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactcgtctaaaagattttaagaaaggtttcgatatgacctctgctgtgccatcaaatacgaaaaaaaagctggtgattgcttccgatcacgcagcatttgagttgaaatcaaccttgattacttggctgaaagagcttggtcatgaggtcgaagaccttggccctcatgaaaaccattcagtcgattatcccgattacggttataagctggctgtcgctatcgcagaaaaaaccgctgatttcggtattgctttatgtggctcgggaatcggtatctcgatcgctgtcaatcgccatccggctgcccgttgcgctttgattacggataaccttaccgcccgtttggcaagagaacataacaatgccaatgttatcgctatgggtgcgagattgatcggcattgaaaccgctaaggattgtatttcagctttccttgcaacgccgtttggaggtgaacgtcatgttcgccgtatcgataaactttcgaatcctcagttcaatatctagataagttaggagaataaacatgagtaaattacccctgattgctccctctatcctttcggcggattttgcccatttgggagatgaggtcgcggcgatagatcaggccggtgccgattggatccatattgatgtgatggatggccatttcgtgccgaatatcaccataggccccatggttgtgaaggctttgcgtccctatagccaaaagccttttgatgtccatttgatgattgcgcctgtcgatcaatatatcgaggctttttctgaagcgggtgctgatattatcagtttccatcccgaagcgggcgcgcatccccatcgcactattcagcatatcaaatcattgggcaaaaaagcgggattagtttttaatccggcgacccctttaagctggcttgattatctaatggatgatcttgatctgattatggtgatgagcgttaaccccggttttggcggccaaaaatttatcaaaacccaattagaaaagattaaagatatccgtcaaagaattaccgcctctgggcgggatatccgcttggaagtggatggcggaattgatgccacgactgcaccgcttgccgtcgaagccggtgccgatgttttggtcgcgggaacggccagctttaaaggcggcgcaacatgttacaccgataatatcaggatattgcgtaaatcatgattaattaactcgaggcggcctgaacgtactgcaagtcctgacgtcactgtgcagtccgttggcccggttatcggtagcgataccgggcatttttttaaggaacgatcgatagaattcgcggccggcccggcaagacgtgatatggaaccggaatttgctccggcattcctgcgcaaagatagctaatatctttcatattttgtatcgaaaaaggagggtctttaaagatcctccttttttttgcataaaaagaaggccatagaacaaacagtgataaagacagtctcaaactgtctttttatagaaaataccagaatattgtatctgggggaggatgcatggtcttaatccggaataccccggtcatgcacaggatgttagagcttttgcctttatggcaaaataaaccatggctcgggaatatctgcgctttgatttttgtaggatgtgccttccttgtccgtagtattattgggcattttttaccggcaggttatcctttcgtgacctttatgccgacaatgcttgtggttactttcctctttgggacaagaccgggtattatcgcggctattcttagcttgatggttgcgccttattttatcgaagaaggaagccgatttaacggtgtattggtctggtttctttgcctgctagaaacagtcactgatatgggattggtgattgcgctacagcaaggtaattaccgcctccagaaaaagcgtgcctataatcagatgctggctgaacgcaatgagttgctgtttcatgaattacagcatcgcatttcaaataacttacaggttattgcgtcattattgcggatgcaaagccgcagcatcaccgatgaaaaagccaaggaagctattgatgcctctgttcgtcggattcatatgatcggtgaattacagcgggcgctttatattaaaaacgggaatcagcttggggcaaaattgatccttgatcgcttgatcaaagaggtcattgcgtccagtaatctcccgaacatccgctataaaatagaagctgaagacctgatcttaccgtcagatatggcaatccctttagcgcttgtatctgctgaatccgtttcaaacgcgttagagcatggctttaaaggcgatcataaagacgcgtttattgaaattaagcttcaaaaaattagcgggcaaatcgaacttaccatttccaataatggcaaacctcttccccaaggcttttcccttgaaaaggtcgatagcttaggcctgaaaattgcggctatgtttgcccgacaattcaaaggaaaattcaccttaagtaatcagcctaaccgttatgtggtttctagccttattttgccttgcggttaggcggccgcctaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgatgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtcgacctttgtagtcttggcctgttgtgtgcatgagcaaatcaatggcaccaccccctcctttttgagctgaatggtcataaaatttataattatctatcgtaattcggaatctatgttcagggtctcgccattgctttttgtctgctgggtcaagttccatgcctaaggtttttaagacatcagaaagaggtattgcacgcatgctatcagcttttcttctagctaatgacagggcttcctctgctctatctgctcgttttttttcttccacatatctcgccgctttgtcagccagcggctgtattacggaaagtgccgatttttgggcttttaggcgttctttttctgcccattcttccttatttgtaaaaattgagggtgggatgggtgcctgaatcttgggatctagctgtaaagttttgttgatatttccgtaatgtctttggactctttgatgcgttgcttttgaaccttttacgcctctggccagccctagaggctccatagaagccgcataatccgtctggagggcagaaagggcttttcgaccatcaaaccatctcgatgcgtttaaacggcctgtatcggggtctctaggcaccataaagccggttaagtggggtgttgtttcatcagcatgtagctgaagagatacaaggttgttttctccaaaggtttgttccgcccattgctgggtgattgttttccagtgttcgagtttttcaggagtggcctgttttgaccattctggagacataccaaagaacagttctatggcctgcacaccgttttttctaagaggctttcccgtttctttctgaattttattcagcatagatttaacatctgctgatgggtcagtagagcctttgagtatttcgtttagttcttttctatctgggtcagcgttttgtgtttcgcggcctcgcgtcatatgcaggctcgcggctttaatcgtgccaactgttttatgtttttcaaacctaaagattgcatagttcggcatgttttaactgctttaatttgagaaaagaccagaggaaataatccagcctatatttctttccctagtagcgaactggaattgtttttccgaaggaaaaaagcaattccgtagtgagtactgaatttattctgattcgtcttgcttttggagcgtctttttgcgttctataactgttgtgaaagctacgcggtcgccattgaaaacgaaattaggattaataaaataccatccttggcgaacatgctttgcaatgattttagctttttctaattcggctagacctcttgcaaaggtagcttgagatagtgccagttttttttcttgtgcgttaagaaagtcctctaaaacgaatttgtctaaagggacgaggtctttgctgatgcctttgtcttgaagtatccaaaccagaacgctgaaagcttttattccagcggctcctagttcaaaagttagcgcgatattggtgctaaataattttacaaattcttcactatcaacacgtctgtaagtcgtcacatgagtgccttgcatctcaccagtggcttgattgaccagaatgttatcatctcgtcctaatcgagataactgaaccctctgacttttaactggcacaaccataccttcgatgaaaggattctcgtcatatctgattggctgctttctcaattttgtcgccatatttgataaacctttaatcaaaaaaaccacattttttgattatacctattcatcgaatgaggcaaggtctatcaattttacccctttttttgatagacggtttaatcaatattgatagaccccttcacagattctgaaaatcgacttccctattttagggatattttcacgattccctttcttagttcttcctagtggggaaattcgttgaatcctgcctcggaaaaaccatgagaaagctgttggttatatacacgggcaaagccaccctatttttagctactggggaaagagataaggcagggtatttgtaaaattaaaaccggatttttcgctttacggtttgtttaggcgcaactgtctttttaagaccgcgtttaaccatcaaaagatcgttccaatcttttccgtgtatcatctgttctttaggtgggagccagttttcaactttttttgttggaaacgcggctttaatcgctccgactaatagcgatgctgctctttgtcctacagcatcccaatcataggcaatatggacagaagatgccttttcaacgatttttcggagagttttagtaagagacgttcttacgccgctggtgcttaataattttacgccagctttaattttttctgggcttaaaaagccgactactgaaatcgcgtctatcgcactttcagcgatataaagatcatacttttcgtcattttttacattgatgctgccagtaaaatgggcttcgcgactgcttcccaaggctaaccctttaaaaccactgcttgttccgcgtaattctgcgccctgaagtgtatctttatcgtcatacatcaagaaggctacattaccgcgatcatctgttcggatagagtcaggaatattgttaaatgatattcctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgggagctccctatcgtctgactcgcaaggctgaacgtgttgacgccttgagcaaggccaaagcggttcttgacgaagccttcccagaagctgatccgacagaaaagctgcgcatccagaagcttgcgaagaagctggaagcaaaaatcgtccgcaccgccattctgaaagaaggccggagaattgacggacgcgatctgaaaacagttcgcccgatccgctctcaggttggattcttgccccgcacgcatggttctgccctgtttacgcgtggtgaaacacaggctttggtttcaaccacccttggaacggcggatgctgaacagatgatcgacggtttaaccggccttcattatgaacgcttcatgctgcattacaacttccccccatattcggtcggtgaagttggtcgttttggtgctccgggtcgtcgtgaaatcggccatggtaaactggcatggcgtgcgcttcatccggttttgccgagcaaggctgatttcccgtataccatccgtgttttgtcggatatcaccgaatctaatggttcctcttccatggcaaccgtttgcggtggctgccttgcattgatggatgccggtgttcccttaacgcgtccggtttccggtatcgccatgggtcttattctggaaaaagacggcttcgctattttgtccgatatcatgggtgatgaagatcacttgggtgatatggactttaaggtcgccggtaccgaaaaaggtatcaccagcctccagatggacatcaaggttgctggcattaccgaagaaatcatgcagaaagctttggaacaggctaaaggtggccgtgctcatatcttgggtgaaatgtccaaagcgctgggtgaagtccgctccgaaatttctaatttggcaccgcgcattgaaacaatgagcgtaccaaaagacaaaatccgtgatgttatcggaacgggcggaaaagttatccgtgaaatcgtggcgaccacaggtgccaaggtcgatatcgaagatgacggcacggttcgtctgtcttcttctgatccggccaatattgaagcagcccgtgaatggatcaatggtattgttgaagaaccggaagtaggcaaaatctataacggtaaagtcgtcaatatcgttgatttcggtgccttcgtaaacttcatgggtggccgtgacggcttggtacatgtttcggaaatcaagaacgaacgtgtcaacaaggtcagcgatgtcctgtccgaaggtcaggaagtcaaagtcaaggttcttgaaattgacaaccgtggcaaggttcgcctgtctatgcgtgttgtcgatcaggaaaccgga

SEQ ID NO: 30 - DNA, 1318 bp, artificial sequence
  constructed fragment for integration into Z. m pnp gene
cctatcgtctgactcgcaaggctgaacgtgttgacgccttgagcaaggccaaagcggttcttgacgaagccttcccagaagctgatccgacagaaaagctgcgcatccagaagcttgcgaagaagctggaagcaaaaatcgtccgcaccgccattctgaaagaaggccggagaattgacggacgcgatctgaaaacagttcgcccgatccgctctcaggttggattcttgccccgcacgcatggttctgccctgtttacgcgtggtgaaacacaggctttggtttcaaccacccttggaacggcggatgctgaacagatgatcgacggtttaaccggccttcattatgaacgcttcatgctgcattacaacttccccccatattcggtcggtgaagttggtcgttttggtgctccgggtcgtcgtgaaatcggccatggtaaactggcatggcgtgcgcttcatccggttttgccgagcaaggctgatttcccgtataccatccgtgttttgtcggatatcaccgaatctaatggttcctcttccatggcaaccgtttgcggtggctgccttgcattgatggatgccggtgttcccttaacgcgtccggtttccggtatcgccatgggtcttattctggaaaaagacggcttcgctattttgtccgatatcatgggtgatgaagatcacttgggtgatatggactttaaggtcgccggtaccgaaaaaggtatcaccagcctccagatggacatcaaggttgctggcattaccgaagaaatcatgcagaaagctttggaacaggctaaaggtggccgtgctcatatcttgggtgaaatgtccaaagcgctgggtgaagtccgctccgaaatttctaatttggcaccgcgcattgaaacaatgagcgtaccaaaagacaaaatccgtgatgttatcggaacgggcggaaaagttatccgtgaaatcgtggcgaccacaggtgccaaggtcgatatcgaagatgacggcacggttcgtctgtcttcttctgatccggccaatattgaagcagcccgtgaatggatcaatggtattgttgaagaaccggaagtaggcaaaatctataacggtaaagtcgtcaatatcgttgatttcggtgccttcgtaaacttcatgggtggccgtgacggcttggtacatgtttcggaaatcaagaacgaacgtgtcaacaaggtcagcgatgtcctgtccgaaggtcaggaagtcaaagtcaaggttcttgaaattgacaaccgtggcaaggttcgcctgtctatgcgtgttgtcgatcaggaaaccgg

SEQ ID NO: 31 - DNA, 1225 bp, artificial sequence
  constructed fragment for integration into the Z. m pnp gene
cggcaagacgtgatatggaaccggaatttgctccggcattcctgcgcaaagatagctaatatctttcatattttgtatcgaaaaaggagggtctttaaagatcctccttttttttgcataaaaagaaggccatagaacaaacagtgataaagacagtctcaaactgtctttttatagaaaataccagaatattgtatctgggggaggatgcatggtcttaatccggaataccccggtcatgcacaggatgttagagcttttgcctttatggcaaaataaaccatggctcgggaatatctgcgctttgatttttgtaggatgtgccttccttgtccgtagtattattgggcattttttaccggcaggttatcctttcgtgacctttatgccgacaatgcttgtggttactttcctctttgggacaagaccgggtattatcgcggctattcttagcttgatggttgcgccttattttatcgaagaaggaagccgatttaacggtgtattggtctggtttctttgcctgctagaaacagtcactgatatgggattggtgattgcgctacagcaaggtaattaccgcctccagaaaaagcgtgcctataatcagatgctggctgaacgcaatgagttgctgtttcatgaattacagcatcgcatttcaaataacttacaggttattgcgtcattattgcggatgcaaagccgcagcatcaccgatgaaaaagccaaggaagctattgatgcctctgttcgtcggattcatatgatcggtgaattacagcgggcgctttatattaaaaacgggaatcagcttggggcaaaattgatccttgatcgcttgatcaaagaggtcattgcgtccagtaatctcccgaacatccgctataaaatagaagctgaagacctgatcttaccgtcagatatggcaatccctttagcgcttgtatctgctgaatccgtttcaaacgcgttagagcatggctttaaaggcgatcataaagacgcgtttattgaaattaagcttcaaaaaattagcgggcaaatcgaacttaccatttccaataatggcaaacctcttccccaaggcttttcccttgaaaaggtcgatagcttaggcctgaaaattgcggctatgtttgcccgacaattcaaaggaaaattcaccttaagtaatcagcctaaccgttatgtggtttctagccttattttgccttgcggttag

SEQ ID NO: 32 - DNA, 25 bp, artificial sequence
  primer
cggcttcaatcggattgttagcagg

SEQ ID NO: 33 - DNA, 38 bp, artificial sequence
  primer
cgtgtagcttggacactcatgtttattctcctaactta

SEQ ID NO: 34 - DNA, 20 bp, artificial sequence
  synthetic primer
ccagtatcagcccgtcatac

SEQ ID NO: 35 - DNA, 20 bp, artificial sequence
  synthetic primer
ccagcatggttgtgatggct

SEQ ID NO: 36 - DNA, 20 bp, artificial sequence
  synthetic primer
gccttgggcttttaaagcct

SEQ ID NO: 37 - DNA, 22 bp, artificial sequence
  synthetic primer
gagaagggttggttgtggcatc

SEQ ID NO: 38 - DNA, 22 bp, artificial sequence
  synthetic primer
gtggatggcggaattgatgcca

SEQ ID NO: 39 - DNA, 26 bp, artificial sequence
  synthetic primer
tctcggagagatagaggtcagtcgac

SEQ ID NO: 40 - DNA, 23 bp, artificial sequence
  synthetic primer
cagctatgatgacagcgcattgg

SEQ ID NO: 41 - DNA, 23 bp, artificial sequence
  synthetic primer
gggcggttcgatccatagaaagg

SEQ ID NO: 42 - DNA, 2247 bp, Zymomonas mobilis
atgttcgatattaaacgccaggaaatcgattggggcggaaaaaaactgacactggaaaccggacaggttgcccgtcaggcagatggcgccgtcattgcgaccttaggtgaaacggtcgtattatgcgcggtaacggcagcaaaaacggtaaaagaaggtcaggatttctttcctttgaccgtccattatcaggaaaaatattcagcagcaggccgtattcccggtggctttttcaagcgtgaacgtggcgcaaccgaacgggaaacgctgatttcacggttaatcgaccgtccaatccgtcctctgtttccggaaggtttctataacgaaaccttggtcattgcgcaggtcatgtcctatgacggcgagaatgaaccggatatcttggcgatgatcgctgcttctgcggctcttgctctttccggtgtgcctttcttgggccccatcggtgctgcccgtgtgggttatcaagatggcgagttcattcttaacccgaccttggaacagcttgaaaaaagtgatcttgatctggttgtcggggctacccgtgatgccgtgatgatggttgaatcggaagcgaatgagcttcccgaagaagtcatgctcaatgccgtttcttttgcgcatgaatctttacagccggttatcaaagctatcatcaatctggcagaacaggccgctaaagagccttgggaactggtcagctatgatgacagcgcattggctgccaaagtcgaagaactctgctacgacaatttcgataaggcctatcgtctgactcgcaaggctgaacgtgttgacgccttgagcaaggccaaagcggttcttgacgaagccttcccagaagctgatccgacagaaaagctgcgcatccagaagcttgcgaagaagctggaagcaaaaatcgtccgcaccgccattctgaaagaaggccggagaattgacggacgcgatctgaaaacagttcgcccgatccgctctcaggttggattcttgccccgcacgcatggttctgccctgtttacgcgtggtgaaacacaggctttggtttcaaccacccttggaacggcggatgctgaacagatgatcgacggtttaaccggccttcattatgaacgcttcatgctgcattacaacttccccccatattcggtcggtgaagttggtcgttttggtgctccgggtcgtcgtgaaatcggccatggtaaactggcatggcgtgcgcttcatccggttttgccgagcaaggctgatttcccgtataccatccgtgttttgtcggatatcaccgaatctaatggttcctcttccatggcaaccgtttgcggtggctgccttgcattgatggatgccggtgttcccttaacgcgtccggtttccggtatcgccatgggtcttattctggaaaaagacggcttcgctattttgtccgatatcatgggtgatgaagatcacttgggtgatatggactttaaggtcgccggtaccgaaaaaggtatcaccagcctccagatggacatcaaggttgctggcattaccgaagaaatcatgcagaaagctttggaacaggctaaaggtggccgtgctcatatcttgggtgaaatgtccaaagcgctgggtgaagtccgctccgaaatttctaatttggcaccgcgcattgaaacaatgagcgtaccaaaagacaaaatccgtgatgttatcggaacgggcggaaaagttatccgtgaaatcgtggcgaccacaggtgccaaggtcgatatcgaagatgacggcacggttcgtctgtcttcttctgatccggccaatattgaagcagcccgtgaatggatcaatggtattgttgaagaaccggaagtaggcaaaatctataacggtaaagtcgtcaatatcgttgatttcggtgccttcgtaaacttcatgggtggccgtgacggcttggtacatgtttcggaaatcaagaacgaacgtgtcaacaaggtcagcgatgtcctgtccgaaggtcaggaagtcaaagtcaaggttcttgaaattgacaaccgtggcaaggttcgcctgtctatgcgtgttgtcgatcaggaaaccggcgcagagctggatgataaccgtccgccacgtgagaacgcagaacgtcgcggtggtgagcgtcctcgtcgtgatcggggccctcgtcgggaatctggcgatcgtccggcaagacgtgatatggaaccggaatttgctccggcattcctgcgcaaagatagctaa

SEQ ID NO: 43 - DNA, 474 bp, artificial sequence
  start codon changed to ATG
atgacctctgctgtgccatcaaatacgaaaaaaaagctggtgattgcttccgatcacgcagcatttgagttgaaatcaaccttgattacttggctgaaagagcttggtcatgaggtcgaagaccttggccctcatgaaaaccattcagtcgattatcccgattacggttataagctggctgtcgctatcgcagaaaaaaccgctgatttcggtattgctttatgtggctcgggaatcggtatctcgatcgctgtcaatcgccatccggctgcccgttgcgctttgattacggataaccttaccgcccgtttggcaagagaacataacaatgccaatgttatcgctatgggtgcgagattgatcggcattgaaaccgctaaggattgtatttcagctttccttgcaacgccgtttggaggtgaacgtcatgttcgccgtatcgataaactttcgaatcctcagttcaatatctag

SEQ ID NO: 44 - DNA, 39 bp, artificial sequence
  primer
catcttactgcggccgcgtgacggaagatcacttcgcag

SEQ ID NO: 45 - DNA, 37 bp, artificial sequence
  primer
tcactcatttaattaacttattcaggcgtagcaccag

SEQ ID NO: 46 - DNA, 1002 bp, artificial sequence
  constructed fragment that contains a Cmr-cassette that is flanked by two wild type loxP sites
ccataacttcgtatagcatacattatacgaagttatttaattaacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacgcggccgcataacttcgtatagcatacattatacgaagttatgcgat

SEQ ID NO: 47 - DNA, 36 bp, artificial sequence
  primer
ctactcatcctgcaggcttctcggtgatcgtgttgc

SEQ ID NO: 48 - DNA, 38 bp, Artificial sequence
  primer
tcactcatggccggccgaacagatcgacggtattgatg

SEQ ID NO: 49 - DNA, 37 bp, Artificial sequence
  primer
catcttactgcgatcgcgatcaatcgcccgatgaatg

SEQ ID NO: 50 - DNA, 36 bp, Artificial sequence
  primer
catcttactggcgcgcctcgccgtattgtatcgctg

SEQ ID NO: 51 - DNA, 20 bp, Artificial sequence
  primer
ctacttcacttcatgaccgg

SEQ ID NO: 52 - DNA, 35 bp, Artificial sequence
  primer
agtcatgcaggcctctgatgaatgctcatccggaa

SEQ ID NO: 53 - DNA, 20 bp, Artificial sequence
  primer
gtctgacgttgatcctgatc

SEQ ID NO: 54 - DNA, 38 bp, artificial sequence
  primer
tcactcatggccggcctgcgtataatatttgcccatgg

SEQ ID NO: 55 - DNA, 21 bp, artificial sequence
  primer
gttcctgctttgcttttgtgg

SEQ ID NO: 56 - DNA, 22 bp, artificial sequence
  primer
cccggaagctatcaaaattttg

SEQ ID NO: 57 - DNA, 18 bp, artificial sequence
  primer
caccgtagtggccgacac

SEQ ID NO: 58 - DNA, 21 bp, artificial sequence
  primer
gatggttttgcacatcagcag

SEQ ID NO: 59 - DNA, 20 bp, artificial sequence
  primer
tctgcaccgataggattggg

SEQ ID NO: 60 - DNA, 21 bp, artificial sequence
  primer
gatgtctttgtctatttcgcg

SEQ ID NO: 61 - DNA, 20 bp, artificial sequence
  primer
ccaacgacttcttcagcatg

SEQ ID NO: 62 - DNA, 20 bp, artificial sequence
  primer
cccaatcctatcggtgcaga

SEQ ID NO: 63 - DNA, 21 bp, artificial sequence
  primer
cgcgaaatagacaaagacatc

SEQ ID NO: 64 - DNA, 20 bp, artificial sequence
  primer
catgctgaagaagtcgttgg

SEQ ID NO: 65 - DNA, 23 bp, artificial sequence
  primer
ccaaacaagcttgcatagttgcc

SEQ ID NO: 66 - DNA, 20 bp, artificial sequence
  primer
gcagatggtggttgagcata

SEQ ID NO: 67 - DNA, 20 bp, artificial sequence
  primer
tgccattttgtggaacgacg

SEQ ID NO: 68 - DNA, 19 bp, artificial sequence
  primer
tgcagttggtgtgggaatg

SEQ ID NO: 69 - DNA, 21 bp, artificial sequence
  primer
ggtttgactcatcaacatggc

SEQ ID NO: 70 - DNA, 20 bp, artificial sequence
  primer
cgtcgttccacaaaatggca

SEQ ID NO: 71 - DNA, 19 bp, artificial sequence
  primer
cattcccacaccaactgca

SEQ ID NO: 72 - DNA, 21 bp, artificial sequence
  primer
gccatgttgatgagtcaaacc

(19) United States
(12) Patent Application Publication
(10) Pub. No.: US 2013/0157332 A1
(43) Pub. Date: Jun. 20, 2013

(54) GENE INACTIVATION ALLOWING IMMEDIATE GROWTH ON XYLOSE MEDIUM BY ENGINEERED ZYMOMONAS

(71) Applicant: E I Du Pont De Nemours And Company, Wilmington, DE (US)

(72) Inventors: WILLIAM D. HITZ, Wilmington, DE (US); Min Qi, Hockessin, DE (US); Luan Tao, Wallingford, PA (US); Paul V. Viitanen, West Chester, PA (US); Jianjun Yang, Hockessin, DE (US)

(73) Assignee: E I Du Pont De Nemours And Company, Wilmington, DE (US)

(21) Appl. No.: 13/711,647

(22) Filed: Dec. 12, 2012

Related U.S. Application Data
(60) Provisional application No. 61/577,879, filed on Dec. 20, 2011.

(51) Int. Cl. C12N 15/74

(52) U.S. Cl. CPC C12N 15/74
USPC 435/161; 435/471; 435/252.3

(57) ABSTRACT

Zymomonas cells that are genetically engineered to have a disrupted aldose reductase gene such that aldose reductase activity for conversion of xylose to xylitol in the presence of NADPH is reduced by greater than 90%, and that are engineered to express a xylose utilization metabolic pathway, were found to have the ability to gro on medium containing xylose as the only sugar without adaptation in media containing xylose.

This application claims the benefit of U.S. Provisional Application 61/577,879, filed Dec. 20, 2011, and is incorporated by reference in its entirety.

### FIELD OF THE INVENTION

The invention relates to the fields of microbiology and genetic engineering. More specifically, inactivation of a gene in the Zymomonas genome that is annotated as encoding an aldo/keto reductase was found to allow immediate growth on xylose by Zymomonas engineered to express a xylose utilization pathway without a xylose-adaptation step.

### BACKGROUND OF THE INVENTION

Production of ethanol by microorganisms provides an alternative energy source to fossil fuels and is therefore an important area of current research. It is desirable that microorganisms producing ethanol, as well as other useful products, be capable of using xylose as a carbon source since xylose is the major pentose in hydrolyzed lignocellulosic biomass. Biomass can provide an abundantly available, low cost carbon substrate. Zymomonas mobilis and other bacterial ethanologens which do not naturally utilize xylose have been genetically engineered for xylose utilization by introduction of genes encoding 1) xylose isomerase, which catalyses the conversion of xylose to xylulose; 2) xylulokinase, which phosphorylates xylulose to form xylulose 5-phosphate; 3) transketolase; and 4) transaldolase (U.S. Pat. No. 5,514,583, U.S. Pat. No. 5,712,133, U.S. Pat. No. 6,566,107, WO 95/28476, Feldmann et al. (1992) Appl. Microbiol. Biotechnol. 38: 354-361, Zhang et al. (1995) Science 267:240-243; Yanase et al. (2007) Appl. Environ. Mirobiol. 73:2592-2599). Typically the coding regions used were from E. coli genes.

Even with expression of this xylose utilization pathway however, engineered strains of Zymomonas usually require an adaptation period in xylose-containing medium before they are able to grow on xylose when it is the sole carbon source. Strains engineered for expression of the xylose utilization pathway have been adapted by serial passage on xylose-containing medium, resulting in strains with improved xylose utilization as described in U.S. Pat. No. 7,223,575 and U.S. Pat. No. 7,741,119. Disclosed in U.S. Pat. No. 7,989,206 is the finding that during adaptation, the Zymomonas mobilis glyceraldehyde-3-phosphate dehydrogenase gene promoter expressing E. coli xylose isomerase was mutated to a more active form that increased the level of xylose isomerase activity, which had previously been the rate-limiting enzyme for xylose metabolism.

U.S. Pat. No. 7,741,119 also discloses improved xylose utilization by inactivation of the gfor locus encoding glucose-fructose oxidoreductase, an enzyme that is able to generate xylitol when glucose andxylulose are both available. Thus, in growth media that contained glucose and xylose, about 3-fold more xylitol was produced by Zymomonas cells that express xylose isomerase, which converts xylose to xylulose, as compared to cells lacking xylose isomerase (U.S. Pat. No. 7,741,119). Moreover, this increase did not occur when the GFOR gene was inactivated, thus demonstrating the importance of this enzyme in the production of xylitol in vivo.

It has been established using cell-free extracts that a wild type strain of Z. mobilis (CP4) has NADPH-dependent aldose reductase activity that can directly convert D-xylose to xylitol (Feldmann et al. Appl. Microbiol. Biotechnol. (1992) 38:354-361). Another wild type strain of Z. mobilis (ZM4) was also reported to have NADPH-dependent aldose reductase activity (Agrawal et al. 2011 108:777-785). In that study a plasmid that contained all four genes that are required for xylose metabolism was introduced into ZM4, and the resulting transformants could not grow on xylose without an adaptation step. The adapted strain that resulted from this procedure (A1) was able to grow on xylose, but only very slowly. To improve xylose utilization, A1 was further adapted in a process that took 80 days and 30 serial transfers with xylose as sole carbon source. The new adapted strain (A3) grew better on xylose than the A1 parent. It also produced less xylitol, was more resistant to xylitol, and had higher xylose isomerase activity. Thus at least three different mutations took place during the evolution of the A3 strain. Although the number of mutations that occurred during the first adaptation period that resulted in the A1 strain, (which was able to grow on xylose, albeit poorly), was not determined, the authors noted that both adapted strains, A1 and A3, had “barely detectable” NADPH-dependent aldose reductase activities compared to wild type ZM4 carrying an empty plasmid. It was subsequently reported that the A3 strain has a point mutation in the coding region of the ZMO0976 gene, which codes for an enzyme that has NADPH-dependent xylose reductase activity that is able to convert xylose to xylitol (Agrawal and Chen (2011) Biotechnol. Lett. 33:2127-2133). The purified mutant protein has <5% of the activity of the wild type enzyme, based on expression in E. coli. It was also shown in the same study that benzaldehyde and furfural are better substrates for the ZMO0976 gene product than xylose.

There remains a need for engineered strains of Zymomonas and other bacterial ethanolagens that contain the genes for xylose utilization and are able to grow on media that contains xylose as sole carbon source without a preliminary adaptation step, and processes for using these strains to produce ethanol.

### SUMMARY OF THE INVENTION

The invention provides Zymomonas cells that are genetically engineered to be competent to grow on medium containing xylose as the only sugar without adaptation, and methods for making said cells and for using said cells for ethanol production.

Accordingly, the invention provides a method of making a xylose-competent Zymomonas cell comprising:
a) providing a Zymomonas host cell;
b) creating a genetic modification in said cell in at least one endogenous gene encoding an aldose reductase enzyme of EC 1.1.1.21 having the ability to convert xylose to xylitol in the presence of NADPH; and
c) introducing into said cell a xylose utilization metabolic pathway;
   wherein the order of steps (b) and (c) is not specified and wherein said xylose-competent Zymomonas cell has aldose reductase activity reduced by greater than 90% as compared with a Zymomonas cell lacking the genetic modification of step (b).

In another embodiment the invention provides a xylose-competent and ethanol-producing Zymomonas cell having the following characteristics:
a) the cell does not require a xylose-adaptation step for immediate growth on xylose-containing media;
b) the cell has aldose reductase activity for conversion of xylose to xylitol in the presence of NADPH reduced by greater than 90%; and
c) the cell comprises a xylose utilization metabolic pathway comprising a series of polynucleotides encoding polypeptides, each having xylose isomerase, xylulokinase, transketolase, or transaldolase enzymatic activity.

Additionally the invention provides a method for producing ethanol comprising growing the xylose-competent and ethanol-producing Zymomonas cell of the invention under conditions wherein ethanol is produced.

### BRIEF DESCRIPTION OF THE DRAWINGS AND SEQUENCE DESCRIPTIONS

FIG. 1 shows a diagram of metabolic pathways for xylose utilization and ethanol production.

FIG. 2 shows a diagram of the first two steps of the engineered xylose utilization pathway (boxed), xylitol synthesis, xylitol 5-phosphate formation (a toxic dead-end intermediate), and inhibition of xylose isomerase by xylitol.

FIG. 3 shows plasmid maps of pZX21 (A), pZX52 (B), and pZX6 (C).

FIG. 4 shows graphs of growth, xylose used, and ethanol produced for cultures grown in mRM3-X10 of ZW1-109 (A), ZW1-210 (B), and control ZW1 (C).

FIG. 5 shows a plasmid map of pMODlinker-Cm.

FIG. 6 (A) shows a plasmid map of pAR-cm and (B) shows a diagram of primer binding sites in a portion of the ZW658 chromosome that includes the ZMO0976 ORF.

FIG. 7 shows a diagram of a region of the ZW1 chromosome that includes the ZMO0976 ORF following a double-crossover event with the pAR-cm suicide construct, with primer binding sites and PCR products marked.

FIG. 8 shows a graph of xylitol production by two ZMO0976 knockout mutants (AR1, AR2) and the control wild type Z. mobilis ZW1.

FIG. 9 shows a graph of growth in medium containing 100 g/L of xylose as the only sugar, monitored by OD600, of Z. mobilis strains engineered to express a xylose utilization pathway (on pZB4) either with knockout of the ZMO0976 gene (AR1; three isolates: -1, -2, -3) or with the native ZMO0976 gene (ZW1; three isolates: -A, -B, -C). The filled symbols are all overlapping.

FIG. 10 shows a graph of growth in medium containing 100 g/L of xylose as the only sugar, monitored by OD600, of eight Z. mobilis isolates engineered to express a xylose utilization pathway (on pZB4) after adaptation in medium containing 45 g/L xylose and 5 g/L glucose, and then in medium that contained 100 g/L xylose as the only sugar. The three types of adapted mutant strains that were isolated (Groups #1-3) are indicted by the circles.

The invention can be more fully understood from the following detailed description and the accompanying sequence descriptions which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the nucleotide sequence of the ZMO0976 coding region.

SEQ ID NO:2 is the amino acid sequence of the protein encoded by the ZMO0976 coding region.

SEQ ID NO:3 is the nucleotide sequence of the ZmPgap from the CP4 strain of Z. mobilis.

SEQ ID NO:4 is the nucleotide sequence of the improved Pgap from strain ZW658 which is called the 801 GAP promoter or the Super GAP promoter or P_{gapS}.

SEQ ID NO:5 is the nucleotide sequence of the improved Pgap from strain 8b.

SEQ ID NO:6 is the nucleotide sequence of an improved Pgap with both position 116 (ZW658) and position 217 (8b) mutations in the pZB4 variant of Pgap.

SEQ ID NO:7 is the nucleotide sequence of an improved Pgap with the position 116 mutation from ZW658 in the CP4 variant of Pgap.

SEQ ID NO:8 is the nucleotide sequence of an improved Pgap with the position 217 mutation from 8b in the CP4 variant of Pgap.

SEQ ID NO:9 is the nucleotide sequence of an improved Pgap with both position 116 (ZW658) and position 217 (8b) mutations in the CP4 variant of Pgap.

SEQ ID NO:10 is the nucleotide sequence of an improved Pgap with the position 116 mutation from ZW658 in the ZM4 variant of Pgap.

SEQ ID NO:11 is the nucleotide sequence of an improved Pgap with the position 217 mutation from 8b in the ZM4 variant of Pgap.

SEQ ID NO:12 is the nucleotide sequence of an improved Pgap with both position 116 (ZW658) and position 217 (8b) mutations in the ZM4 variant of Pgap.

SEQ ID NO:13 is the coding region for the Actinoplanes missourinesis xylose isomerase that was codon optimized for Zymomonas.

SEQ ID NO:14 is the nucleotide sequence of the ZMO0976 coding region with a 78 nucleotide deletion from strains ZW641 and ZW658.

SEQ ID NO:15 is the nucleotide sequence of plasmid pZX21.

SEQ ID NO:16 is the nucleotide sequence of the GFO-L fragment.

SEQ ID NO:17 is the nucleotide sequence of the gfor coding sequence.

SEQ ID NO:18 is the nucleotide sequence of the GFO-R fragment.

SEQ ID NO:19 is the nucleotide sequence of a 1,661-bp chimeric xylA gene containing the 304-bp Z. mobilis Super GAP promoter, a 1,185-bp A. missouriensis xylA coding sequence, and a 166-bp E. coli araD 3′UTR with a 5′ XbaI site.

SEQ ID NO:20 is the nucleotide sequence of a 1,960-bp chimeric xylB gene containing a 191 bp Pₑₙₒ, a 1,455-bp E. coli xylB coding sequence and a 314-bp E. coli xylB 3′UTR.

SEQ ID NO:21 is the nucleotide sequence of a 1,014 bp aadA marker (for spectinomycin resistance; Spec-R) bounded by lox sites.

SEQ ID NO:22 is the nucleotide sequence of shuttle vector pZX52.

SEQ ID NO:23 is the nucleotide sequence of the LDH-L fragment.

SEQ ID NO:24 is the nucleotide sequence of the LDH-R fragment.

SEQ ID NO:25 is the nucleotide sequence of the IdhA coding sequence.

SEQ ID NO:26 is the nucleotide sequence of a 3,339 bp P_{gapT}-Tal-Tkt operon containing a 304-bp T-mutant of the Z. mobilis GAP promoter (P_{gapT}), a 954-bp E. coli Tal coding region, a 1,992-bp E. coli Tkt coding region, and a 68-bp E. coli Tkt 3′UTR.

SEQ ID NO:27 is the nucleotide sequence of the P_{gapT} promoter.

SEQ ID NO:28 is the nucleotide sequence of a 1,443 bp Pₑₙₒ-Rpi-Rpe operon containing a 191 bp Pₑₙₒ, a 471 bp Z. mobilis Rpi coding sequence, a 663 bp Z. mobilis Rpe coding sequence, and a 35 bp E. coli xylA 3′UTR.

SEQ ID NO:29 is the nucleotide sequence of the DCO shuttle vector pZX6.

SEQ ID NO:30 is the nucleotide sequence of the PNP-L fragment.

SEQ ID NO:31 is the nucleotide sequence of the PNP-R fragment.

SEQ ID NOs:32 to 41 are PCR primers.

SEQ ID NO:42 is the nucleotide sequence of the pnp coding region from Zymomonas mobilis strain ZM4.

SEQ ID NO:43 is the nucleotide sequence of the coding region for the Z. mobilis RPI protein with the start codon mutated to ATG.

SEQ ID NOs:44 and 45 are primers.

SEQ ID NO:46 is the nucleotide sequence of Fragment 2 containing a Cm^{r}-cassette that is flanked by two wild type loxP sites

SEQ ID NOs:47 to 72 are primers.

### DETAILED DESCRIPTION

The following definitions may be used for the interpretation of the claims and specification:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

“Gene” refers to a nucleic acid fragment that expresses a specific protein or functional RNA molecule, which may optionally include regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

“Promoter” or “Initiation control regions” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”.

The term “expression”, as used herein, refers to the transcription and stable accumulation of coding (mRNA) or functional RNA derived from a gene. Expression may also refer to translation of mRNA into a polypeptide. “Overexpression” refers to the production of a gene product in transgenic organisms that exceeds levels of production in normal or non-transformed organisms.

The term “transformation” as used herein, refers to the transfer of a nucleic acid fragment into a host organism, resulting in genetically stable inheritance. The transferred nucleic acid may be in the form of a plasmid maintained in the host cell, or some transferred nucleic acid may be integrated into the genome of the host cell. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “genetic modification” refers, non-inclusively, to any modification, mutation, base deletion, base addition, codon modification, gene over-expression, gene suppression, promoter modification or substitution, gene addition (either single or multicopy), antisense expression or suppression, or any other change to the genetic elements of a host cell or bacterial strain, whether they produce a change in phenotype or not.

The term “recombinant bacterial host cell” refers to a bacterial cell that comprises at least one heterologus gene or genetic construct or nucleic acid fragment.

The term “NADPH-dependent xylose reductase activity” refers to aldose reductase activity for conversion of xylose to xylitol in the presence of NADPH.

The term “xylose-competent Zymomonas cell” refers to a Zymomonas cell that is able to grow in medium containing xylose as the only sugar, without being adapted for growth on xylose.

The term “adapted for growth on xylose” refers to a cell or strain isolated after prolonged growth in medium containing xylose. Adaptation may include a period of growth in medium containing xylose and glucose, and then a period of growth in medium containing only xylose, each medium being a xylose-containing medium. Typically the prolonged period of growth is at least about four days.

The term “xylose metabolic pathway” or “xylose utilization metabolic pathway” refers to a series of enzymes (encoded by genes) that metabolize xylose through to fructose-6-phosphate and/or glyceraldehyde-6-phosphate and include 1) xylose isomerase, which catalyses the conversion of xylose to xylulose; 2) xylulokinase, which phosphorylates xylulose to form xylulose 5-phosphate; 3) transketolase; and 4) transaldolase.

The term “xylose isomerase” refers to an enzyme that catalyzes the interconversion of D-xylose and D-xylulose. Xylose isomerases (XI) belong to the group of enzymes classified as EC 5.3.1.5.

The term “ribose-5-phosphate isomerase” or “RPI” refers to an enzyme that catalyzes the interconversion of ribulose-5-phosphate and ribose-5-phosphate. Ribose-5-phosphate isomerases belong to the group of enzymes classified as EC 5.3.1.6.

The term “ribulose-phosphate 3-epimerase” or “RPE” refers to an enzyme catalyzes the interconversion of D-ribulose 5-phosphate and D-xylulose 5-phosphate and is classified as EC 5.1.3.1.

The term “carbon substrate” or “fermentable carbon substrate” refers to a carbon source capable of being metabolized by microorganisms. A type of carbon substrate is “fermentable sugars” which refers to oligosaccharides and monosaccharides that can be used as a carbon source by a microorganism in a fermentation process.

The term “lignocellulosic” refers to a composition comprising both lignin and cellulose. Lignocellulosic material may also comprise hemicellulose.

The term “cellulosic” refers to a composition comprising cellulose and additional components, including hemicellulose.

The term “saccharification” refers to the production of fermentable sugars from polysaccharides.

The term “pretreated biomass” means biomass that has been subjected to thermal, physical and/or chemical pretreatment to increase the availability of polysaccharides in the biomass to saccharification enzymes.

“Biomass” refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat, wheat straw, barley straw, hay, rice straw, switchgrass, waste paper, sugar cane bagasse, sorghum, components obtained from milling of grains, trees, branches, roots, leaves, wood chips, sawdust, shrubs and bushes, vegetables, fruits, flowers and animal manure.

“Biomass hydrolysate” refers to the product resulting from saccharification of biomass. The biomass may also be pretreated or pre-processed prior to saccharification.

The term “heterologous” means not naturally found in the location of interest. For example, a heterologous gene refers to a gene that is not naturally found in the host organism, but that is introduced into the host organism by gene transfer. For example, a heterologous nucleic acid molecule that is present in a chimeric gene is a nucleic acid molecule that is not naturally found associated with the other segments of the chimeric gene, such as the nucleic acid molecules having the coding region and promoter segments not naturally being associated with each other.

As used herein, an “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

The term “complementary” is used to describe the relationship between nucleotide bases that are capable of hybridizing to one another. For example, with respect to DNA, adenosine is complementary to thymine and cytosine is complementary to guanine.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign program of the LASERGENE bioinformatics computing suite (DNASTAR Inc., Madison, Wis.).

Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign v8.0 program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992); Thompson, J. D. et al, Nucleic Acid Research, 22 (22): 4673-4680, 1994) and found in the MegAlign v8.0 program of the LASERGENE bioinformatics computing suite (DNASTAR Inc.). Default parameters for multiple alignment (stated as protein/nucleic acid (GAP PENALTY=10/15, GAP LENGTH PENALTY=0.2/6.66, Delay Divergen Seqs(%)=30/30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 50%, 55%, 60%, 65%, 70%, 75%, 80%, 85%, 90%, or 95%, or any integer percentage from 50% to 100% may be useful in identifying polypeptides of interest, such as 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above identities but typically encode a polypeptide having at least 50 amino acids, preferably at least 100 amino acids, and more preferably at least 125 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et. al., Short Protocols in Molecular Biology, 5^{th} Ed. Current Protocols. John Wiley and Sons, Inc., N.Y., 2002.

The present invention relates to methods of creating strains of Zymomonas that can grow in medium containing xylose as the only sugar directly following introduction of a xylose utilization pathway, without an adaptation period in xylose-containing medium. Xylose is one of the predominant pentose sugars in hydrolyzed lignocellulosic materials, making utilization of xylose desirable for fermentation of biomass hydrolysate. Biomass hydrolysate can provide an abundant, renewable carbohydrate resource for biocatalytic production of target products. Eliminating an adaptation step in xylose-containing medium, which is time-consuming and where multiple undefined mutations may occur, greatly facilitates development of a xylose-utilizing biocatalyst.

### Engineered Xylose Utilizing Zymomonas

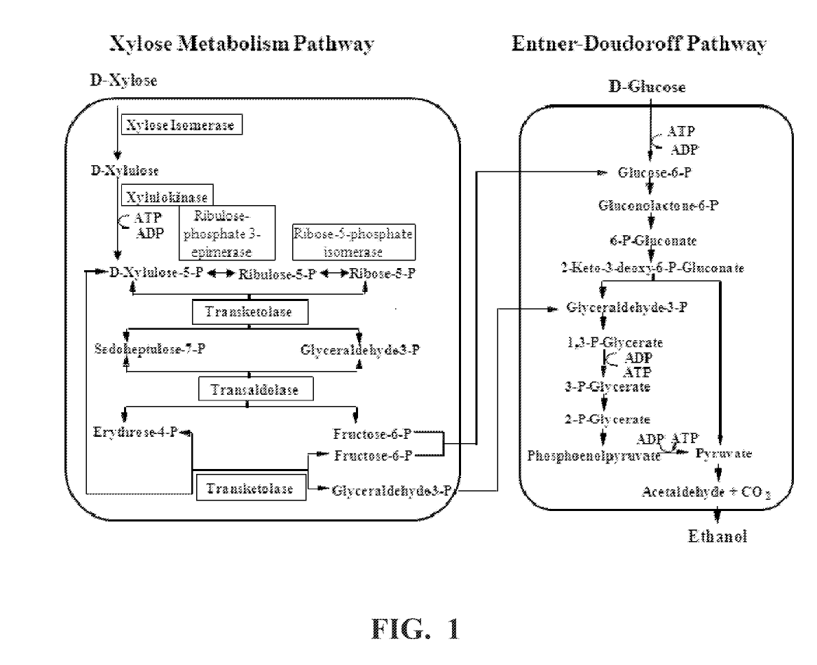

Zymomonas cells naturally produce ethanol using glucose, fructose and/or sucrose as fermentation substrates, but xylose is not metabolized. Strains of ethanol-producing Zymomonas, such as Z. mobilis have been engineered for xylose fermentation to ethanol. Typically four genes have been introduced into Z. mobilis for expression of four enzymes involved in xylose metabolism to create a xylose utilization metabolic pathway (FIG. 1) as described in U.S. Pat. No. 5,514,583, U.S. Pat. No. 5,712,133, U.S. Pat. No. 6,566,107, WO 95/28476, Feldmann et al. ((1992) Appl Microbiol Biotechnol 38: 354-361), and Zhang et al. ((1995) Science 267:240-243). These include genes encoding xylose isomerase which catalyzes the conversion of xylose to xylulose, and xylulokinase which phosphorylates xylulose to form xylulose 5-phosphate. Additionally expressed are transketolase and transaldolase, two enzymes of the pentose phosphate pathway that convert xylulose 5-phosphate to intermediates that couple pentose metabolism to the glycolytic Entner-Douderoff pathway permitting the metabolism of xylose to ethanol (see FIG. 1). DNA sequences encoding these enzymes may be obtained from any of numerous microorganisms that are able to metabolize xylose, such as enteric bacteria, and some yeasts and fungi. Sources for the coding regions may include Xanthomonas, Klebsiella, Escherichia, Rhodobacter, Flavobacterium, Acetobacter, Gluconobacter, Rhizobium, Agrobacterium, Salmonella, Pseudomonads, and Zymomonas. The coding regions of E. coli are typically used.

The encoding DNA sequences are operably linked to promoters that are expressed in Zymomonas cells such as the promoter of Z. mobilis glyceraldehyde-3-phosphate dehydrogenase (GAP promoter), and Z. mobilis enolase (ENO promoter). A mutant GAP promoter with increased expression as disclosed in U.S. Pat. No. 7,989,206, which is incorporated herein by reference, is also useful for expression in Zymomonas. The coding regions may individually be expressed from promoters, or two or more coding regions may be joined in an operon with expression from the same promoter. The resulting chimeric genes may be introduced into Zymomonas cells and maintained on a plasmid, or integrated into the genome using, for example, homologous recombination, site-directed integration, or random integration. Examples of strains engineered to express a xylose utilization metabolic pathway include CP4(pZB5) (U.S. Pat. No. 5,514,583), ATCC31821/pZB5 (U.S. Pat. No. 6,566,107), 8b (US 20030162271; Mohagheghi et al., (2004) Biotechnol. Lett. 25; 321-325), and ZW658 (ATTCC #PTA-7858).

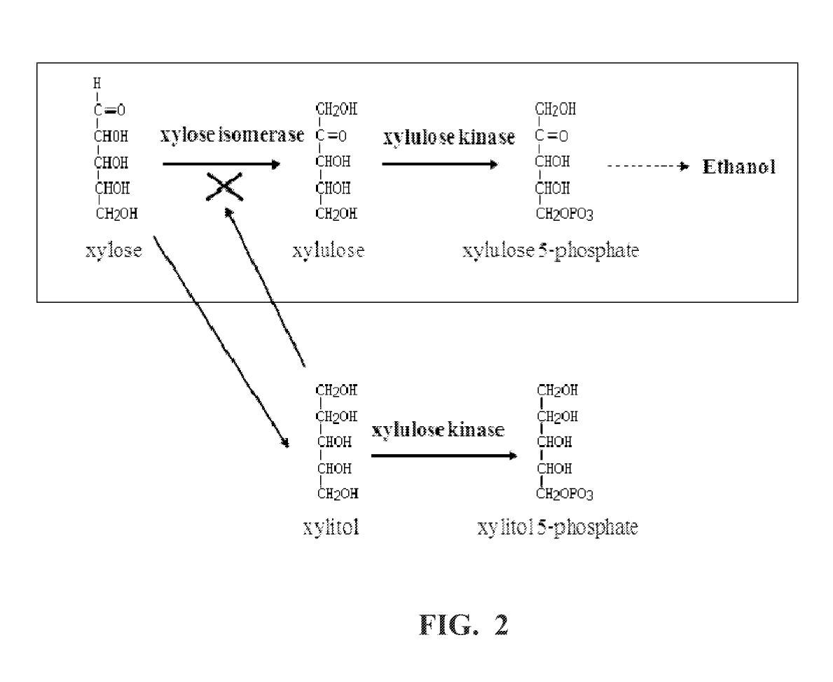

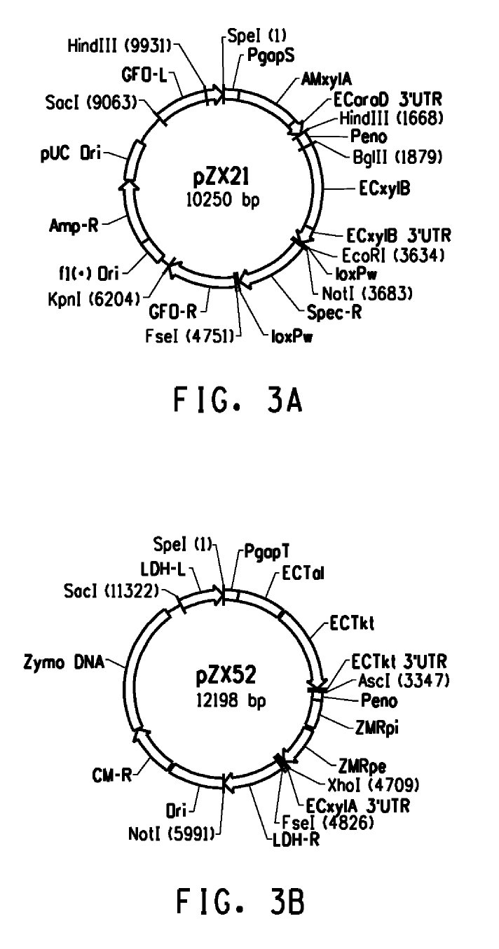

Strains engineered to express the xylose utilization metabolic pathway as described above typically are not able to immediately grow in medium containing xylose as the only sugar. One metabolic issue for these strains is a side pathway shown in FIG. 2 (outside the box) that not only reduces efficiency of ethanol production from xylose (boxed section), but may also have a detrimental effect on cell viability in the presence of xylose, especially when xylose is the sole carbon source. In this pathway, xylose is converted to xylitol, which in turn is converted to xylitol 5-phosphate by xylulose kinase which is the second enzyme in the engineered xylose utilization metabolic pathway. Xylitol 5-phosphate is a toxic compound that inhibits bacterial growth, and it has clearly been demonstrated that E. coli xylulokinase is directly responsible for the inhibitory effect of xylitol through its conversion to xylitol 5-phosphate (Akinterinwa et al. (2009) Metabolic Engineering 11: 48-55). In addition, conversion of xylose to xylitol also reduces substrate flow to ethanol, and xylitol is also an inhibitor of xylose isomerase, the first enzyme in the engineered pathway for xylose utilization. Experiments have established that there are at least two different pathways for xylitol formation in Z. mobilis: 1) by glucose-fructose oxidoreductase (U.S. Pat. No. 7,741,119), and 2) by NADPH-dependent aldose reductase activity (Feldmann et al. supra; Agrawal et al. (2011) Biotechnology and Bioengineering 108:777-785).

Cells of Zymomonas that are engineered for expression of the xylose utilization metabolic pathway generally require a period of adaptation in xylose-containing medium prior to being able to grow in medium that contains xylose as the only sugar. During the adaptation step, which is typically a prolonged period of serial transfers in xylose-containing medium, multiple mutations can occur in the genome. A number of specific mutations which have occurred during adaptation of different strains that were engineered for xylose utilization have been identified, but it is not clear which ones are important and/or necessary for the initial ability to grow on xylose as sole carbon source. Indeed, prior to this work, no single mutation had been identified that allows immediate growth of a Zymomonas strain expressing the xylose utilization metabolic pathway in medium containing xylose as the only sugar.

### Engineering Immediate Growth in Xylose Medium

The present invention relates to a new finding that genetic modification affecting expression of a single enzyme activity can be made in cells of Zymomonas that also are engineered for xylose utilization, which allows immediate growth in medium containing xylose as the only sugar, without an adaptation step. These cells are xylose-competent Zymomonas cells. The genetic modification disrupts the expression of at least one endogenous gene encoding an NADPH-dependent aldose reductase that is able to convert xylose to xylitol, thereby causing a reduction of NADPH-dependent xylose reductase activity in cell-free extracts by greater than 90%. The activity may be reduced by greater than 90%, 91%, 92%, 93%, 94%, 95%, 96%, 97%, 98%, or 99%. NADPH-dependent xylose reductase activity is measured in cell-free extracts by monitoring the conversion of NADPH to NADP at 340 nm in the presence of xylose as described in the General Methods herein.

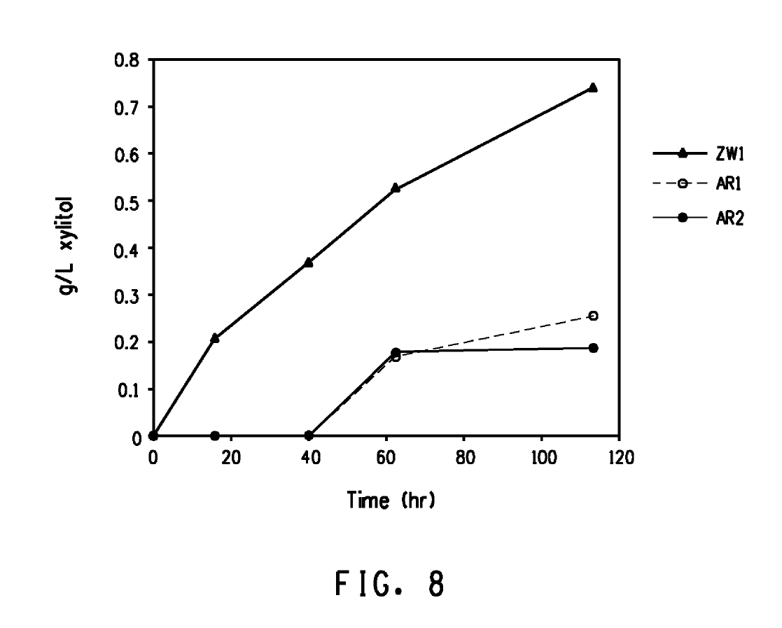

Reducing NADPH-dependent xylose reductase activity by greater than 90% in otherwise wild type Zymomonas cells, by disrupting expression of an endogenous gene encoding a putative aldo/keto reductase (as designated by Seo et al. (2005) Nat. Biotechnol. 23: 63-68) was found herein to reduce production of xylitol by the cells when grown in xylose-containing medium. Even with this genetic manipulation, however, cellular production of xylitol was not completely eliminated, although it was reduced by about a factor of three (shown in FIG. 8).

Cells with this disruption, that were subsequently provided with the four enzymes that are required for xylose utilization (a xylose utilization metabolic pathway), were shown herein (Example 6) to be able to immediately grow in medium containing xylose as the only sugar without the need for an adaptation step. Thus these are xylose-competent Zymomonas cells which can be selected for using medium containing only xylose as the carbon source. Alternatively, in a Zymomonas cell that already has the four enzymes that are required for xylose-utilization but has not been adapted for growth on xylose, intentional inactivation of NADPH-dependent xylose reductase activity can be selected for using medium that only contains xylose as the carbon source.

Immediate growth on media that contained only xylose without an adaptation step was enabled even in the presence of wild type glucose-fructose oxidoreductase (GFOR) activity. GFOR strongly contributes to xylitol production in Zymomonas cells engineered to express xylose isomerase, the first enzyme in the xylose metabolism pathway, since it converts xylose to xylulose. In contrast to the direct reduction of xylose to xylitol by NADPH-dependent xylose reductases, xylitol production by GFOR only occurs in the presence of glucose-containing media when a source of xylulose is also present as disclosed in U.S. Pat. No. 7,741,119, which is incorporated herein by reference.

In the present method, expression of one or more endogenous aldose reductase encoding genes of the Zymomonas genome is disrupted. In the fully sequenced genome of Zymomonas there are multiple genes annotated as encoding aldo/keto reductases. For example, in the sequenced genome of the wild type Zymomonas mobilis ZM4 strain (GenBank accession number AE008692; Seo et al., Nat. Biotechnol. 23 (1), 63-68 (2005)), there are at least four genes that code for putative aldo/keto reductases, which are named ZMO0976, ZMO1344, ZMO1673, and ZMO1773. The proteins encoded by the ZMO1344, ZMO1673, and ZMO1773 genes have 24%, 29% and 38% amino acid sequence identities, respectively, to the ZMO0976 encoded protein (SEQ ID NO:2). Two additional genes recently re-annotated as aldo/ketose reductases (ZMO062 and ZMO1984) encode proteins with about 32% identity to the ZMO976 encoded protein. Aldo/keto reductases belong to a superfamily of soluble NAD(P)H oxidoreductases whose chief purpose is to reduce aldehydes and ketones to primary and secondary alcohols. Aldose reductases, also called aldehyde reductases, are classified as EC 1.1.1.21. The present method and strains concern aldose reductase enzymes that are able to convert xylose to xylitol in the presence of NADPH (which is converted to NADP), an enzyme activity which is referred to herein as NADPH-dependent xylose reductase activity.

A genetic modification causing disruption of only the ZMO0976 gene in Z. mobilis strain ZW1 is shown herein in Example 5 to reduce NADPH-dependent xylose reductase activity by greater than 90%. Thus in the present method, the wild type Z. mobilis strain ZM4 (ATCC #31821; ZW1 is another name for ZM4) and derivatives thereof are engineered with a genetic modification that disrupts the ZMO0976 gene, which is identified in the ZM4 genomic sequence of GenBank accession number AE008692. This is the only genetic modification needed to provide competency for growth on medium containing xylose as the only sugar when the four xylose pathway enzymes are introduced into wild type ZM4, eliminating the need for a xylose-adaptation step. The other genes annotated as encoding aldo/keto reductases in the ZM4 genome (GenBank accession number AE008692), ZMO1344, ZMO1673, ZMO177, ZM0062, and ZMO1984 did not significantly contribute to NADPH-dependent aldose reductase activity in the presence of xylose in cell-free extracts, as shown in Example 5 herein.

The ZMO0976 gene has the coding sequence of SEQ ID NO:1, which encodes a protein of SEQ ID NO:2 shown herein to have NADPH-dependent xylose reductase activity. Wild type strains Z. mobilis NCIMB 11163 and ATCC 10988 have genes with coding regions that have 100% nucleotide sequence identity to SEQ ID NO:1, and encode proteins that have 100% amino acid sequence identity to SEQ ID NO:2. These genes with identical nucleotide sequences in Z. mobilis strains NCIMB 11163 and ATCC 10988 are disrupted in the present method to provide competency for growth on medium containing xylose as the only sugar, with no adaptation for growth on xylose.

There may be variation in sequences of the genes that are equivalent to ZMO0976 in different strains of Zymomonas, which may be genetically modified in the present method. Thus coding sequences with nucleotide sequence identity of at least about 95%, 96%, 97%, 98%, or 99% to SEQ ID NO:1 may represent the ZMO0976 gene in different strains of Zymomonas. In addition, proteins with amino acid sequence identity of at least about 95%, 96%, 97%, 98%, or 99% to SEQ ID NO:2 may represent the ZMO0976 gene product in different strains of Zymomonas. In the present methods and strains, genes having coding regions with these identities, or genes encoding proteins with these identities, are disrupted.

In other Zymomonas species or strains it may be necessary to inactivate multiple genes that code for proteins that have NADPH-dependent xylose reductase activity to achieve the greater than 90% reduction of total NADPH-dependent aldose reductase enzyme activity that allows immediate growth on xylose. One of skill in the art can readily determine by experimentation, using genetic modification methods described below and others known in the art, which gene or genes annotated as aldo/keto reductase in a Zymomonas genome sequence should be disrupted to obtain greater than 90% decrease in NADPH-dependent xylose reductase activity.

Genetic modification to disrupt an NADPH-dependent xylose reductase encoding gene described above, that is a target gene, in the present method may be using any method known to one skilled in the art such as methods that affect its expression of mRNA or protein, or the function or stability of the encoded protein. Genetic modifications may be, for example, mutation, insertion, or deletion in the coding region, or other region of the gene such as the promoter. Methods include, but are not limited to, deletion of the entire or a portion of the gene, inserting a DNA fragment into the gene (in either the promoter or coding region) so that the encoded protein cannot be expressed, introducing a mutation into the coding region which adds a stop codon or frame shift such that a functional protein is not expressed, and introducing one or more mutations into the coding region to alter amino acids so that a non-functional protein is expressed. All of these methods may be readily practiced by one skilled in the art making use of the known target NADPH-dependent xylose reductase coding sequence (such as SEQ ID NO:1), as well as the Zymomonas DNA sequence that surrounds the target NADPH-dependent xylose reductase coding sequence, such as that which is available in the complete Z. mobilis genome sequence (GenBank Accession AE008692).

A particularly suitable method for creating a genetic modification in an NADPH-dependent xylose reductase encoding target gene, as exemplified herein in Examples 3 and 4, is using double-crossover homologous recombination mediated by ZMO0976 flanking DNA sequences bounding a chloramphenicol resistance or other marker gene, leading to insertion of the marker gene in the target aldose reductase coding region such that a functional protein is not expressed. In addition, the marker gene may be bounded by site-specific recombination sites, so that following expression of the corresponding site-specific recombinase, the resistance gene is excised from the gene. The site-specific recombination leaves behind a recombination site which disrupts expression of the aldose reductase encoding gene. The homologous recombination vector may be constructed to also leave a deletion in the aldose reductase encoding gene following excision of the marker, as is well known to one skilled in the art.

In one embodiment the present method steps (b) and (c) are performed concurrently by integration of one or more genes of the xylose utilization metabolic pathway into the endogenous target gene encoding NADPH-dependent xylose reductase, thereby disrupting its expression. In other embodiments integration of one or more genes of the xylose utilization metabolic pathway may be in another gene, thereby creating an additional genetic modification as described above.

For any of the host cells and during any of the steps of the present method there is no need for a preliminary adaptation process to achieve immediate growth on medium that contains xylose as the sole carbon source, provided the engineered xylose metabolism pathway provides sufficient carbon flux to support growth. No additional mutations, other than disruption of at least one endogenous gene encoding NADPH-dependent xylose reductase activity that reduces activity by greater than 90%, are required in a host cell engineered with a xylose utilization metabolic pathway to provide said growth property. However, even with said disruption, further adaptation in xylose-containing media can result in better growth on xylose through natural selection of other mutations that increase carbon flux through the engineered xylose pathway. In one embodiment said disruption is only in the ZMO0976 gene (such as in GenBank accession number AE008692; such as in SEQ ID NO:1).

Additional Genetic Modifications

In one embodiment a wild type strain of Zymomonas is used as the host cell for introduction of a xylose utilization metabolic pathway and a genetic modification disrupting expression of at least one endogenous gene encoding an NADPH-dependent xylose reductase. In various embodiments the host cell or xylose-competent Zymomonas cell has one or more additional genetic modifications that are performed prior to, concurrently with, or after steps (b) and/or (c) of the present method. Additional genetic modifications may include any modification that improves the strain such as one that increases growth rate and/or cell mass, increases utilization of xylose and/or allows the use of other sugars, increases tolerance to inhibitory compounds such as acetate, or increases production of ethanol. These genetic modifications can be introduced through rational design or they can occur spontaneously through random mutations and natural selection by further adaptation of the strain in various xylose-containing growth media.

In one embodiment Zymomonas cells may be additionally engineered for arabinose utilization which is described in U.S. Pat. No. 5,843,760, which is incorporated herein by reference. To allow arabinose utilization, genes expressed in addition to genes of the xylose utilization pathway include: 1) L-arabinose isomerase to convert L-arabinose to L-ribulose, 2) L-ribulokinase to convert L-ribulose to L-ribulose-5-phosphate, and 3) L-ribulose-5-phosphate-4-epimerase to convert L-ribulose-5-phosphate to D-xylulose (U.S. Pat. No. 5,843,760). As disclosed in US 2011/0143408, which is incorporated herein by reference, improved arabinose utilization may be achieved by additionally expressing an arabinose-proton symporter, such as by expressing a coding region from an araE gene.

In another embodiment the endogenous himA gene, which encodes the alpha subunit of the integration host factor, is genetically modified to reduce its expression which improves growth in medium containing acetate as described in U.S. Pat. No. 7,897,396, which is incorporated herein by reference. Acetate is present in biomass hydrolysate, thus when using medium containing biomass hydrolysate, increased tolerance to this component is desired.

In another embodiment a genetic modification is made that reduces glucose-fructose oxidoreductase (GFOR) activity as described in U.S. Pat. No. 7,741,119, which is incorporated herein by reference. Reduced expression of GFOR, as well as of the himA gene, may be by any method such as those described above for reducing aldose reductase activity.

In another embodiment a genetic modification is made which increases ribose-5-phosphate isomerase (RPI) activity, as disclosed in commonly owned and co-pending U.S. patent application Ser. No. 13/161,734, published as US20120156746A1, which is incorporated herein by reference. Increased RPI expression may be accomplished by increasing expression of the endogenous RPI encoding gene, such as with a promoter that is more highly active than the native promoter, or by expressing a heterologous gene encoding any protein or polypeptide with ribose-5-phosphate isomerase activity in Zymomonas. There are two groups of ribose-5-phosphate isomerase enzymes that are called RPI-A and RPI-B, as described in U.S. application Ser. No. 13/161,734, US20120156746A1, either of which may be expressed.

In another embodiment, the xylose isomerase that is expressed as part of the xylose utilization metabolic pathway is expressed using a mutant, highly active promoter that is disclosed in U.S. Pat. No. 7,989,206 and U.S. Pat. No. 7,998,722, which are incorporated herein by reference. Mutant promoters disclosed therein are promoters of the Zymomonas mobilis glyceraldehyde-3-phosphate dehydrogenase gene (Pgap) having a mutation of G to T at position 116 or of C to T at position 217 of the promoter of SEQ ID NO:3. Mutant, more highly active promoters include SEQ ID NOs:4, 5, 6, 7, 8, 9, 10, 11, and 12. These include variant promoter sequences from different strains of Z. mobilis with one or both of the mutations at positions equivalent to 116 and 217 in SEQ ID NO:3.

In another embodiment a xylose isomerase that is expressed as part of the xylose utilization metabolic pathway is a Group I xylose isomerase included in the class of enzymes identified by EC 5.3.1.5 as disclosed in commonly owned and co-pending US Patent Application Publication US2011-0318801. It is disclosed therein that Group I xylose isomerases, such as one expressed from a coding region isolated from Actinoplanes missouriensis (coding region with codon optimization for Zymomonas: SEQ ID NO:13), have higher activity in Zymomonas than Group 2 xylose isomerases. Group I xylose isomerases are defined therein by molecular phylogenetic bioinformatics analysis (using PHYLIP neighbor joining algorithm as implemented in PHYLIP (Phylogeny Inference Package version 3.5c; Felsenstein (1989) Cladistics 5:164-166), GroupSim analysis (Capra and Singh (2008) Bioinformatics 24: 1473-1480), and a Profile Hidden Markov Model (using the hmmsearch algorithm of the HMMER software package; Janelia Farm Research Campus, Ashburn, Va.).

Although no mutation other than one conferring reduction of aldose reductase activity for conversion of xylose to xylitol in the presence of NADPH by greater than 90% is required for immediate growth on xylose, in one embodiment serial transfers of the xylose-competent cells described herein in media containing xylose as a carbon source (adapting in xylose-containing media) may be used to produce new strains that grow better on xylose as a consequence of natural selection through the accumulation of mutations in other genes that are beneficial to xylose metabolism. Adaptation on xylose-containing medium is described in U.S. Pat. No. 7,223,575 and U.S. Pat. No. 7,741,119, which are incorporated herein by reference.

### Characteristics of the Present Cells

The present Zymomonas cells have the natural ability to produce ethanol. In addition the cells, which are grown to provide strains, have greater than 90% reduction in native aldose reductase activity for conversion of xylose to xylitol in the presence of NADPH. In addition the cells have a xylose utilization metabolic pathway comprising a series of polynucleotides encoding polypeptides, each having xylose isomerase, xylulokinase, transketolase, or transaldolase enzymatic activity.

In various embodiments the present strains additionally have one or more characteristic that is an improvement in the strain such as increased growth rate or cell mass production, increased utilization of xylose and/or use of other sugars, increased tolerance to inhibitory compounds such as acetate, or increased production of ethanol. These characteristics are conferred by genetic modifications such as those described above.

### Fermentation for Ethanol Production

An engineered Zymomonas strain having a xylose utilization pathway and at least one disrupted gene encoding aldose reductase, having aldose reductase activity for conversion of xylose to xylitol in the presence of NADPH reduced by greater than 90%, may be used in fermentation to produce ethanol. Zymomonas mobilis is a natural ethanolagen. As an example, production of ethanol by a Z. mobilis strain of the invention is described.

For production of ethanol, recombinant xylose-utilizing Z. mobilis is brought in contact with medium that contains mixed sugars including xylose. Typically the medium contains mixed sugars including arabinose, xylose, and glucose. The medium may contain biomass hydrolysate that includes these sugars that are derived from treated cellulosic or lignocellulosic biomass.

When the mixed sugars concentration is high such that growth is inhibited, the medium includes sorbitol, mannitol, or a mixture thereof as disclosed in U.S. Pat. No. 7,629,156. Galactitol or ribitol may replace or be combined with sorbitol or mannitol. The Z. mobilis grows in the medium where fermentation occurs and ethanol is produced. The fermentation is run without supplemented air, oxygen, or other gases (which may include conditions such as anaerobic, microaerobic, or microaerophilic fermentation), for at least about 24 hours, and may be run for 30 or more hours. The timing to reach maximal ethanol production is variable, depending on the fermentation conditions. Typically, if inhibitors are present in the medium, a longer fermentation period is required. The fermentations may be run at temperatures that are between about 30° C. and about 37° C., at a pH of about 4.5 to about 7.5.

The present Z. mobilis cells may be grown in medium containing mixed sugars including xylose in laboratory scale fermenters, and in scaled up fermentation where commercial quantities of ethanol are produced. Where commercial production of ethanol is desired, a variety of culture methodologies may be applied. For example, large-scale production from the present Z. mobilis strains may be produced by both batch and continuous culture methodologies. A classical batch culturing method is a closed system where the composition of the medium is set at the beginning of the culture and not subjected to artificial alterations during the culturing process. Thus, at the beginning of the culturing process the medium is inoculated with the desired organism and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable for growth of the present Z. mobilis strains and comprise a typical batch system with the exception that the substrate is added in increments as the culture progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH and the partial pressure of waste gases such as CO₂. Batch and Fed-Batch culturing methods are common and well known in the art and examples may be found in Biotechnology: A Textbook of Industrial Microbiology, Crueger, Crueger, and Brock, Second Edition (1989) Sinauer Associates, Inc., Sunderland, Mass., or Deshpande, Mukund V., Appl. Biochem. Biotechnol., 36, 227, (1992), herein incorporated by reference.

Commercial production of ethanol may also be accomplished with a continuous culture. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials as is known to one skilled in the art.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

Particularly suitable for ethanol production is a fermentation regime as follows. The desired Z. mobilis strain of the present invention is grown in shake flasks in semi-complex medium at about 30° C. to about 37° C. with shaking at about 150 rpm in orbital shakers and then transferred to a 10 L seed fermentor containing similar medium. The seed culture is grown in the seed fermentor anaerobically until OD₆₀₀ is between 3 and 6, when it is transferred to the production fermentor where the fermentation parameters are optimized for ethanol production. Typical inoculum volumes transferred from the seed tank to the production tank range from about 2% to about 20% v/v. Typical fermentation medium contains minimal medium components such as potassium phosphate (1.0-10.0 g/l), ammonium sulfate (0-2.0 g/l), magnesium sulfate (0-5.0 g/l), a complex nitrogen source such as yeast extract or soy based products (0-10 g/l). A final concentration of about 5 mM sorbitol or mannitol is present in the medium. Mixed sugars including xylose and at least one additional sugar such as glucose (or sucrose), providing a carbon source, are continually added to the fermentation vessel on depletion of the initial batched carbon source (50-200 g/l) to maximize ethanol rate and titer. Carbon source feed rates are adjusted dynamically to ensure that the culture is not accumulating glucose in excess, which could lead to build up of toxic byproducts such as acetic acid. In order to maximize yield of ethanol produced from substrate utilized, biomass growth is restricted by the amount of phosphate that is either batched initially or that is fed during the course of the fermentation. The fermentation is controlled at pH 5.0-6.0 using caustic solution (such as ammonium hydroxide, potassium hydroxide, or sodium hydroxide) and either sulfuric or phosphoric acid. The temperature of the fermentor is controlled at 30° C.-35° C. In order to minimize foaming, antifoam agents (any class—silicone based, organic based etc) are added to the vessel as needed. An antibiotic, for which there is an antibiotic resistant marker in the strain, such as kanamycin, may be used optionally to minimize contamination.

Any set of conditions described above, and additionally variations in these conditions that are well known in the art, are suitable conditions for production of ethanol by a xylose-utilizing recombinant Zymomonas strain.

### EXAMPLES

### General Methods

Transformation of Z. mobilis

Replicating and non-replicating plasmid DNA was introduced into Z. mobilis using electroporation, essentially as described in U.S. Pat. No. 5,514,583. Briefly, the 50-μl transformation reactions contained ˜10¹⁰ cells/ml in 10% (v/v) glycerol and 1-2 μg of non-methylated plasmid DNA that was isolated from E. coli SCS110. Control reactions were treated identically, but did not receive any plasmid DNA. The settings for the electroporator were 1.6 kv/cm, 200Ω, and 25 μF, and the gap width of the cuvette was 0.1 cm. Following electroporation, the transformation reactions were diluted with MMG medum (50 g/L glucose, 10 g/L yeast extract, 5 g/L of tryptone, 2.5 g/L of (NH₄)₂SO₄, 0.2 g/L K₂HPO₄, and 1 mM MgSO₄) and the cells were allowed to recover at 30° C. before they were plated on MMG medium that contained 1.5% agar (MMG agar plates) with or without antibiotics as indicated. MMX agar plates are identical to MMG agar plates but contain 50 g/L xylose instead of glucose. Plates were incubated in an anaerobic chamber at 30-33° C., until colonies appeared. Additional details are described in the Examples section.

Zymomonas mobilis ZW641 and ZW658 Strain Construction

A detailed description of the construction of the xylose-utilizing recombinant strain, ZW658, starting from the wild type parent strain, ZW1, is provided in U.S. Pat. No. 7,741,084, which is incorporated herein by reference. ZW658 was constructed by integrating two operons, P_{gap}xylAB and P_{gap}taltkt, containing four xylose-utilizing genes encoding xylose isomerase (xylA), xylulokinase (xylB), transaldolase (tal), and transketolase (tkt), with coding regions from the E. coli genes, into the genome of ZW1 (rename of strain ZM4; ATCC 31821) via sequential transposition events followed by adaptation on xylose-containing growth media to produce strain X13L3, which was subsequently renamed ZW641 as disclosed in U.S. Pat. No. 7,989,206, which is incorporated herein by reference. Further adaptation of ZW641 on xylose-containing growth media gave rise to ZW658, which grows much better in xylose and was deposited under the Budapest Treaty as ATCC PTA-7858. As further disclosed in U.S. Pat. No. 7,989,206, ZW658 has higher xylose isomerase activity than ZW641 due to a point mutation in the Z. mobilis P_{gap} promoter that drives the chromosomally integrated E. coli XylA/B operon. This mutant promoter (SEQ ID NO:4), herein called either the 801GAP promoter or the Super GAP promoter or P_{gapS}, has a “T” instead of “G” in position 116, when compared to the native P_{gap} in ZW641 (the 641GAP promoter). The 801Gap promoter is 3- to 4-fold stronger than the 641Gap promoter, and the rate-limiting step for xylose metabolism in ZW641 is xylose isomerase.

### Shake Flask Experiments

Unless otherwise noted, all experiments described below were conducted at 33° C. in shake flasks (15-ml loosely-capped, conical shaped test tubes) using synthetic media that contained glucose and/or xylose as carbon sources. mRM3-G10 medium contains 10 g/L yeast extract, 2 g/L KH₂PO₄, 1 g/L MgSO₄ (7H₂O) and 100 g/L glucose. mRM3-X10 medium is identical but it contains 100 g/L xylose instead of glucose. Cell growth was monitored spectrophometrically by following changes in optical density at 600 nm as a function of time. In the text and figure legends “OD” or “OD600” means optical density at 600 nm. At indicated times during shake flask growth experiments, 1.0-ml aliquots of the cultures were removed for HPLC analysis using an HP 1100 equipped with a refractive index detector (Hewlett-Packard, Palo Alto, Calif.) to determine the concentrations of glucose, xylose, xylitol, ribulose, glycerol, acetate and ethanol that were present in the fermentation broth. Prior to HPLC analysis, cells were removed by centrifugation and the supernatant was filtered through a 0.22 μm cellulose acetate Spin-X centrifuge tube filter (Costar, catalog number 8160) to remove small particles. Compounds were separated on an Aminex HPX-87H column (Bio-Rad) that was run at 55° C. under isocratic conditions using a flow rate of 0.6 ml/min and 0.01 NH₂SO₄ as the mobile phase. Authentic standards of known concentration were used to quantify the peaks of interest and all results are expressed in g/L.

### Preparation of Cell-Free Extracts

Z. mobilis strains ZW1 and ZW1-AR1 were grown in 25 ml of mRM3-G10 to OD ˜2.8, harvested by centrifugation at 3,000×g (4° C.), and cell pellets were rapidly frozen and stored at −80° C. for subsequent use. Cell-free extracts were prepared by mechanical disruption using a Bio101 FastPrep FP120 Cell Disrupter (ThermoSavant); all steps were performed at 0-4° C. except the mechanical disruption step, which was done at room temperature as described below. Frozen cell pellets were thawed, washed three times with 1.0 ml of Lysis Buffer that contained 12.5 mM triethanolamine hydrochloride (adjusted to pH 7.5 with KOH), 0.5 mM EDTA, 1 mM dithiothreitol, and were finally resuspended in 0.8 ml of the same solution. The resuspended cells were transferred to 2.0-ml Lysing Matrix B tubes that contained 0.1 mm silica spheres (MP Biomedicals, Catalog No. 9911-100) and were subjected to three cycles of cell disruption in the FastPrep FP120; each cycle consisted of a 20-sec agitation period at 6 m/sec followed by a 3-min chilling period on ice. The lysate was then centrifuged for 10 min at 20, 800×g and the resulting supernatant was transferred to a fresh tube and re-centrifuged for another 60 min at the same speed. The supernatant from the second centrifugation step is referred to below as the “cell-free extract” in the NADPH-dependent xylose reductase activity assay described below. Protein concentration of the cell-free extracts was measured using the Coomassie (Bradford) Protein Assay Kit (Pierce Biotechnology, Catalog No. 23200) with bovine serum albumin as a standard.

### Measurement of NADPH-Dependent Xylose Reductase Activity

NADPH-dependent aldose reductase activity in cell-free extracts with xylose as the substrate was measured essentially as described (Feldmann et al (1992) Appl Microbial Biotechnol. 38:354-361) with minor modifications. The assay was conducted at 32° C. in a 0.5-ml quartz cuvette that contained the following components: 400 μl of Reaction Buffer (50 mM triethanolamine hydrochloride (pH 7.5) and 5 mM MgSO₄), 40 μl of cell-free extract (that contained 8-10 mg of protein/ml), 10 μl of 10 mM NADPH tetra(cyclohexylammonium) salt and 50 μl of 2M D-xylose. The “no xylose control” reactions were identical, but xylose was omitted and 50 μl of Reaction Buffer was added instead. Enzyme activity was measured spectrophotometrically as a function of time at 340 nm using an extinction coefficient of 6220 M⁻¹cm⁻¹ to monitor the conversion of NADPH to NADP. Enzyme activities are expressed as mU/mg of cell-free extract protein after correcting for the rate of NADPH oxidation in the absence of xylose; 1 mU=1 nanomole of xylitol formed per minute. The signal-to-noise ratio for NADPH-dependent xylose reductase activity was ˜3.5 for the wild type strain, ZW1 (i.e. the rate of NADPH disappearance in the presence of xylose compared to the “no xylose control” reaction).

### Example 1

### Discovery of ZMO0976 Mutation in Adapted Xylose-Utilizing Zymomonas Strains

Comparative genomic sequence analysis of Zymomonas mobilis wild type strain ZW1, which is genetically identical to wild type strain ZM4 (ATCC#31821), and two adapted xylose-utilizing ZW1 derivatives, ZW641 and ZW658 (see General Methods for strain construction) was performed to identify genetic modifications (mutations) that occurred during adaptation on xylose. Whole genome sequence analysis showed that multiple mutations occurred during adaptation of ZW641 and ZW658 strains. Among these mutations was a 78 nucleotide deletion in a coding region of ZW1, which was present in both ZW641 and ZW658. The coding region having the deletion is designated as ZMO0976 in the genomic sequence of the ZM4 strain (GenBank accession number AE008692). This ZMO0976 coding region is annotated as encoding an aldo/keto reductase. The deletion removes a large portion (nucleotides 547 to 624 relative to the start of the initiation codon) of the 1023 nucleotide full coding region (including the stop codon) of ZMO0976 (SEQ ID NO:1), resulting in SEQ ID NO:14.

### Example 2

### Additional Xylose-Utilizing Zymomonas Strains with ZMO0976 Mutations

Two additional xylose-utilizing strains were obtained by introduction of genes for the xylose utilization pathway and adaptation on xylose-containing medium as follows.

Vector Constructs for Building Xylose Utilizing Z. mobilis Strains Using Targeted Integration

A new xylose utilizing Z. mobilis strain was constructed by introducing chimeric xylA, xylB, tal, and tkt genes into the ZW1 strain. The xylB, tal, and tkt coding regions were from E. coli genes as in the ZW658 strain described in General Methods. The xylA coding region was from Actinoplanes missouriensis (AMxylA) which is disclosed in commonly owned and co-pending US Patent Application Publication US2011-0318801, which is incorporated herein by reference, as encoding an enzyme having higher activity than the E. coli xylose isomerase in Z. mobilis. The coding region for the AMxylA was codon optimized for expression in Z. mobilis (SEQ ID NO:13). Additional copies of Z. mobilis rpi and rpe genes were also introduced in order to increase ribose-5-phosphate-isomerase (RPI) and ribulose-phosphate 3-epimerase (RPE) activities. Double crossover (DCO) transformation vectors were designed to specifically integrate the chimeric genes into target regions in Z. mobilis genome.

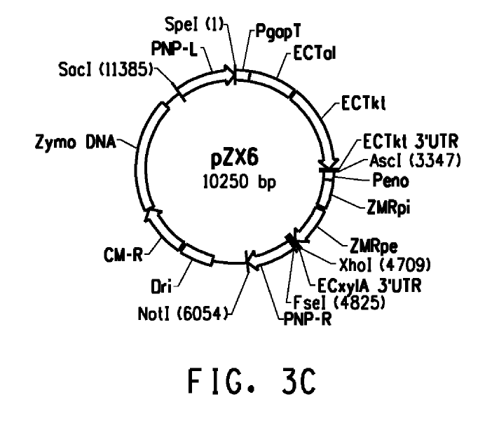

Standard molecular recombination methods were used to construct DCO (double cross over) suicide integration vectors. To express xylose isomerase and xylulose kinase in Z. mobilis, a 10,250-bp DCO suicide vector pZX21 (SEQ ID NO:15; FIG. 3A) was constructed. This vector has a pBluescript backbone which contains a replication origin for E. coli but no Z. mobilis replication origin, thus it cannot be propagated in Z. mobilis making it a suicide vector. It contains DNA sequences from the Z mobilis gene encoding glucose-fructose oxidoreductase, GFO-L and GFO-R, flanking the sequences to be integrated. Both fragments were synthesized by PCR, using Z. mobilis genomic DNA as template. The 1,186-bp GFO-L fragment (SEQ ID NO:16) includes the first 654 bp (from nt-1 to nt-653) of the gfor coding sequence (SEQ ID NO:17) and 533 bp of upstream genomic sequence. The 1,446-bp GFO-R fragment (SEQ ID NO:18) includes the last 480 bp (from nt-823 to nt-1302) of the GFOR coding sequence and 966 bp of downstream genomic sequence. The GFO-L and GFO-R sequences direct integration into the gfor locus, replacing a segment of the gfor coding sequence (from nt-655 to nt-822) in the Z. mobilis genome. This disrupts expression of glucose-fructose oxidoreductase, which reduces xylitol production and increases ethanol production as disclosed in U.S. Pat. No. 7,741,119, which is incorporated herein by reference.

The region in pZX21 between GFO-L and GFO-R includes three chimeric genes. One is a 1,661-bp chimeric xylA gene (SEQ ID NO:19) containing the 304-bp Z. mobilis Super GAP promoter (P_{gapS}; described in U.S. Pat. No. 7,989,206), a 1,185-bp A. missouriensis xylA coding sequence (AMxylA) and a 166-bp E. coli araD 3′UTR with a 5′ XbaI site (ECaraD 3′UTR). The AMxylA coding region was optimized for expression in Z. mobilis according to codon bias of Z. mobilis ZM4 (SEQ ID NO:13). The ECaraD 3′UTR was from the E. coli araBAD operon. The second gene is a 1,960-bp chimeric xylB gene (SEQ ID NO:20) containing a 191 bp Pₑₙₒ, a 1,455-bp E. coli xylB coding sequence (ECxylB) and a 314-bp E. coli xylB 3′UTR (ECxylB 3′UTR). Pₑₙₒ is a strong constitutive promoter from the Z. mobilis genomic DNA having approximately 28% activity of P_{gap}. The third gene is a 1,014 bp aadA marker (for spectinomycin resistance; Spec-R) bounded by lox sites (SEQ ID NO:21). The marker can be removed after integration by expressing Cre recombinase.

To express transaldolase, transketolase, ribose-5-P-isomerase, and D-ribulose-P-3-epimerase in Z. mobilis, a 12,198-bp DCO shuttle vector pZX52 (SEQ ID NO:22; FIG. 3B) was constructed. This vector is a Zymomonas-E. coli shuttle vector which is based on the vector pZB188 (Zhang et al. (1995) Science 267:240-243; U.S. Pat. No. 5,514,583), which includes a 2,582 bp Z. mobilis genomic DNA fragment containing a replication origin allowing the vector to replicate in Zymomonas cells, and a 909-bp E. coli replication origin (Ori). It has a 911 bp chloramphenicol resistance marker (Cm-R) for selection of either E. coli or Z. mobilis transformants. pZX52 contains DNA sequences from the Z mobilis IdhA gene encoding lactate dehydrogenase, LDH-L (875 bp; SEQ ID NO:23) and LDH-R (1,149 bp; SEQ ID NO:24), flanking the sequences to be integrated. These sequences direct integration into the IdhA coding sequence (SEQ ID NO:25) in the Z. mobilis genome between nucleotides #493 and #494, thereby disrupting expression of lactate dehydrogenase.

The region in pZX52 between LDH-L and LDH-R includes two chimeric operons. The first one is a 3,339 bp P_{gapT}-Tal-Tkt operon (SEQ ID NO:26) containing a 304-bp T-mutant of the Z. mobilis GAP promoter (P_{gapT}), a 954-bp E. coli Tal coding region (ECTal), a 1,992-bp E. coli Tkt coding region, and a 68-bp E. coli Tkt 3′UTR (ECTkt 3′UTR). This operon is identical to the naturally existing E. coli Tal-Tkt operon except for the P_{gapT} promoter (SEQ ID NO:27), which is a P_{gap} with a “G” to an “A” change at position 83 and a “T” missing at position 285 as compared to the native P_{gap} (SEQ ID NO:3). The other chimeric operon is a 1,443 bp Pₑₙₒ-Rpi-Rpe operon (SEQ ID NO:28), containing a 191 bp Pₑₙₒ, a 471 bp Z. mobilis Rpi coding sequence with first codon changed to ATG (SEQ ID NO:43) (ZMRpi), a 663 bp Z. mobilis Rpe coding sequence (ZMRpe), and a 35 bp E. coli xylA 3′UTR (ECxylA 3′UTR).

Another DCO shuttle vector named pZX6 (SEQ ID NO:29; FIG. 3C) was constructed. This 12,704 bp vector is a modification of pZX52 having LDH-L and LDH-R sequences replaced with sequences from the Z mobilis pnp gene encoding polynucleotide phosphorylase. The 1,318 bp PNP-L fragment (SEQ ID NO:30) is a segment of the pnp coding sequence (SEQ ID NO:42) from nt-767 to nt-2,084, while the 1,225 bp PNP-R fragment (SEQ ID NO:31) includes the last 59 bp (from nt-2189 to nt-2247) of the pnp coding sequence and 1,166 bp of downstream genomic sequence. Therefore, pZX6 is able to direct integration of the P_{gapT}-Tal-Tkt operon and the Pₑₙₒ-Rpi-Rpe operon into the endogenous pnp gene near the end of the pnp coding sequence and replace a segment of the pnp coding sequence (from nt-2,084 to nt-2,188) in the Z. mobilis genome.

Development of Xylose Utilizing Z. mobilis Strains

The ZW1 strain was transformed with two plasmids in two steps. Competent cells of ZW1 were prepared by growing seed cells overnight in mRM3-G5 (1% yeast extract, 15 mM KH₂PO₄, 4 mM MgSO₄, and 50 g/L glucose) at 30° C. with 150 rpm shaking, to an OD₆₀₀ value near 5. Cells were harvested and resuspended in fresh medium to an OD₆₀₀ value of 0.05. The cells were grown under the same conditions to early to middle log phase (OD₆₀₀ near 0.5). Cells were harvested and washed twice with ice-cold water and then once with ice-cold 10% glycerol. The resulting competent cells were collected and resuspended in ice-cold 10% glycerol to an OD₆₀₀ value near 100. Since transformation of Z. mobilis requires non-methylated DNA, DCO plasmids pZX21, pZX52, and pZX6 were each transformed into E. coli SCS110 competent cells (Stratagene, La Jolla, Calif.). For each transformation, one colony of transformed cells was grown in 10 mL LB-Amp100 (LB broth containing 100 mg/L ampicillin) overnight at 37° C. DNA was prepared from the 10 mL culture, using QIAprep Spin DNA Miniprep Kit (Qiagen).

Approximately 1 μg non-methylated pZX21 DNA was mixed with 50 μL ZW1 competent cells in a 1 MM Electroporation Cuvette (VWR, West Chester, Pa.). The plasmid DNA was electroporated into the cells at 2.0 KV using a BT720 Transporater Plus (BTX-Genetronics, San Diego, Calif.). Transformed cells were recovered in 1 mL MMG5 medium (10 g/L glucose, 10 g/L yeast extract, 5 g/L tryptone, 2.5 g/L (NH₄)₂SO₄, 2 g/L K₂HPO₄, and 1 mM MgSO₄) for 4 hours at 30° C. and grown on MMG5-Spec250 plates (MMG5 with 250 mg/L spectinomycin and 15 g/L agar) for 3 days at 30° C., inside an anaerobic jar with an AnaeroPack (Mitsubishi Gas Chemical, New York, N.Y.).

Since pZX21 is a DCO suicide vector, surviving Spec^{R} colonies had the P_{gapS}-AMxylA::Pₑₙₒ-ECxylB::Spec-R segment integrated into the gfor locus. The colonies were streaked and grown on a fresh MMG5-Spec250 plate, and then subjected to PCR to inspect chimeric gene integration. The first PCR used forward primer ara285 (SEQ ID NO:32) and reverse primer ara120 (SEQ ID NO:33) to inspect double crossover recombination mediated by the GFO-L fragment in pZX21. The ara285 primer matches a segment of Z. mobilis genomic sequence that is 494 bp upstream of the GFO-L fragment in the genome, while ara120 complements the last 18 bp of P_{gapS} and the first 17 bp of AMxylA in pZX21. If integration had occurred as designed, PCR would amplify a 1,903 bp fragment from the transformants. The 2nd PCR used forward primer ara46 (SEQ ID NO:34) and reverse primer ara274 (SEQ ID NO:35) to inspect double crossover recombination mediated by the GFO-R fragment in pZX21. The ara46 primer is a sequence near the end of the Spec^{R} gene in pZX21, while ara274 complements a segment of Z. mobilis genomic DNA that is 83 bp downstream of the GFO-R fragment. This PCR would amplify a 1,864-bp fragment from the colonies having successful integration. Both inspections produced the expected PCR products and thus confirmed accurate transgene integration. The resulting strain was named ZW1-pZX21.

In the second step, ZW1-pZX21 was transformed with pZX52 and selected on a MMG5-Spec250-CM120 (MMG5-Spec250 with 120 mg/L of chloramphenicol) plate. Because pZX52 is a DCO shuttle vector having the Cm^{R} marker for plasmid selection and a markerless integration segment (P_{gapT}-ECTal-ECTkt::Pₑₙₒ-ZMRpi-ZMRpe), the recovered colonies should contain not only the previously integrated construct P_{gapS}-AMxylA::Pₑₙₒ-ECxylB::Spec-R in the Z. mobilis genome, but also the non-integrated construct P_{gapT}-ECTal-ECTkt::Pₑₙₒ-ZMRpi-ZMRpe in the propagated pZX52 plasmid. These transformants should have all required genes for the xylose utilization pathway. To demonstrate that all transgenes were functional in Z. mobilis, ten selected colonies were subjected to a 48-hour growth assay in xylose. In the assay, 2 mL of mRM3-G5-Spec200-CM120 (mRM3-G5 with 200 mg/L spectinomycin and 120 mg/L chloramphenicol) in a 14 mL Falcon polypropylene round-bottom tube was inoculated with a selected colony and cultured overnight at 30° C. with 150 rpm shaking. Tubes were tightly capped, but a hole was punched in the top of the cap using a 23G1 needle for pressure release during cell growth and fermentation. Cells were harvested, washed with MRM3X10 (MRM3 with 100 g/L xylose), and resuspended in mRM3-X10-Spec200-CM120 (mRM3-X10 containing 200 mg/L spectinomycin and 120 mg/L chloramphenicol) to have a starting OD₆₀₀ of 0.1. Five mL of the suspension was placed in a new 14 mL Falcon polypropylene round-bottom tube. Tubes were capped with a hole on the top. Cells were grown for 48 hrs at 30° C. with 150 rpm shaking and OD₆₀₀ was measured on a Shimadzu UV-1201 Spectrophotometer. Then, 1 mL of culture was centrifuged at 10,000×g to remove cells. The supernatant was filtered through a 0.22 μm Costar Spin-X Centrifuge Tube Filter (Corning Inc, Corning, N.Y.) and analyzed for xylose and ethanol by running through a BioRad Aminex HPX-A7H ion exclusion column (BioRad, Hercules, Calif.) with 0.01 NH₂SO₄ at a speed of 0.6 mL/min at 55° C. on an Agilent 1100 HPLC system (Agilent Technologies, Santa Clara, Calif.). Results indicated that all 10 of the transformants had acquired the xylose utilization pathway for ethanol production. The new strain was named ZW1-pZX21-pZX52 and one of the cultures was used in further experiments.

ZW1-pZX21-pZX52 then went through three post-transformation procedures sequentially for integration of the P_{gapT}-ECTal-ECTkt::Pₑₙₒ-ZMRpi-ZMRpe construct.

(1) The strain was adapted on xylose. In this procedure, ZW1-pZX21-pZX52 was suspended in a 5-mL mRM3-G1X9-Spec200-CM120 medium (MRM3 with 10 g/L glucose, 90 g/L xylose, 200 mg/L spectinomycin and 120 mg/L chloramphenicol) with a starting OD₆₀₀ value of 0.2 and grown for 3 to 4 doublings at 30° C. (OD₆₀₀ value from 0.2 to 2; one passage). The culture was then diluted to the starting OD₆₀₀ value and grown for another passage. Totally, 4 passages (approximately 15 doublings) were completed.
(2) Plasmid curing and integration of the P_{gapT}-ECTal-ECTkt::Pₑₙₒ-ZMRpi-ZMRpe construct were carried out by growing 10 μL of the adaptation cell pool in 2 mL mRM3-G5-Spec200 medium at higher temperature (37° C.) for overnight. The 10 μL culture was then diluted in 2 mL mRM3-G5-Spec200 medium and grown for another passage. Totally, 5 passages were performed at 37° C. in glucose medium. As a result of the high temperature growth, the majority of the population should not host the pZP52 plasmid any more, but the P_{gapT}-ECTal-ECTkt::Pₑₙₒ-ZMRpi-ZMRpe construct (lacking a selective marker) should have been integrated into the IdhA gene of the Z. mobilis genome. A minority of the population may maintain pZX52, without integration.
(3) The population was enriched by growing 50 μL of the cell pool in 2 mL mRM3-X10-Spec200 at 30° C. for overnight. The enriched population was grown on a MMG5-Spec250 plate at 30° C. for overnight. Individual colonies were selected and streaked on MMG5 plates and MMG5-CM120 plates in replica. After incubating at 30° C. for overnight, those colonies that grew on MMG5 but not on MMG5-CM120 were selected for further PCR inspection. The first PCR used forward primer ara45 (SEQ ID NO:36) and reverse primer ara356 (SEQ ID NO:37) to inspect double crossover recombination mediated by the LDH-L fragment in pZX52. The ara45 primer matches a segment of Z. mobilis genomic DNA that is 86 bp upstream of the LDH-L fragment in the genome, and ara356 complements a fragment (from nt-91 to nt-112) of the ECTal coding region in pZX52. The PCR would amplify a 1,383-bp fragment from the colonies if integration had occurred as designed. The 2nd PCR used forward primer ara354 (SEQ ID NO:38) and reverse primer ara43 (SEQ ID NO:39) to inspect double crossover recombination mediated by the LDH-R fragment in pZX52. The ara354 primer is a sequence near the 3′ end of ZMRpe in pZX52. The ara43 primer complements a segment of Z. mobilis genomic DNA that is 122 bp downstream of the LDH-R fragment. This PCR would amplify a 1,468 bp fragment from the colonies when recombination was as expected. Both PCRs produced DNA fragments with the expected sizes, which confirmed that the P_{gapT}-ECTal-ECTkt::Pₑₙₒ-ZMRpi-ZMRpe construct had been accurately integrated as designed in all inspected colonies. The resulting colonies were named ZW1-X109.

In a second approach, the ZW1-pZX21 strain was transformed with the pZX6 DCO shuttle vector and the three post-transformation procedures including the xylose-adaptation step were performed as described above for ZW1-X109, except that adaptation was for 10 passages rather than 4 passages. Therefore, the P_{gapT}-ECTal-ECTkt::Pₑₙₒ-ZMRpi-ZMRpe construct was targeted to the endogenous pnp gene. As described for construction of ZW1-X109, the 48-hour growth assay was preformed prior to the three post-transformation procedures to make sure that all transgenes were functioning as expected. After the three post-transformation procedures, the integration was also inspected by PCR. The first PCR used forward primer ara340 (SEQ ID NO:40) and reverse primer ara356 (SEQ ID NO:37) to inspect double crossover recombination mediated by the PNP-L fragment in pZX6. The ara340 primer matches Z. mobilis genomic DNA that is 75 bp upstream of the PNP-L fragment. The ara356 primer used here complements a fragment (from nt-91 to nt-112) of ECTal in pZX6. The PCR produced a 1,815-bp fragment from the transformants, as expected for an accurate integration event. The 2nd PCR used forward primer ara354 (SEQ ID NO:38) and reverse primer ara339 (SEQ ID NO:41) to inspect double crossover recombination mediated by PNP-R fragment in pZX6. In this case, the ara354 primer matches a sequence near the 3′ end of ZMRpe in pZX6, and the ara339 primer complements a segment of Z. mobilis genomic DNA that is 59 bp downstream of the PNP-R fragment sequence. This PCR amplified a 1,549 bp fragment from the transformants, a size that was expected for successful integration. Therefore, PCR inspection confirmed that the P_{gapT}-ECTal-ECTkt::Pₑₙₒ-ZMRpi-ZMRpe construct had been accurately integrated in all inspected colonies. This new strain was named ZW1-X210.

In summary, two xylose utilizing Z. mobilis strains were rebuilt de novo from wild type ZW1. They both had a P_{gapS}-AMxylA::Pₑₙₒ-ECxylB::Spec-R construct integrated into the gfor locus. The ZW1-X109 strain had a P_{gapT}-ECTal-ECTkt::Pₑₙₒ-ZMRpi-ZMRpe construct integrated into the IdhA locus, while the ZW1-X210 strain had the same construct integrated in the endogenous pnp gene. Both strains had one marker gene in the integrated P_{gapS}-AMxylA::Pₑₙₒ-ECxylB::Spec-R construct, which could be removed by introduction of Cre recombinase.

Characterization of New Xylose Utilizing Z. mobilis Strains

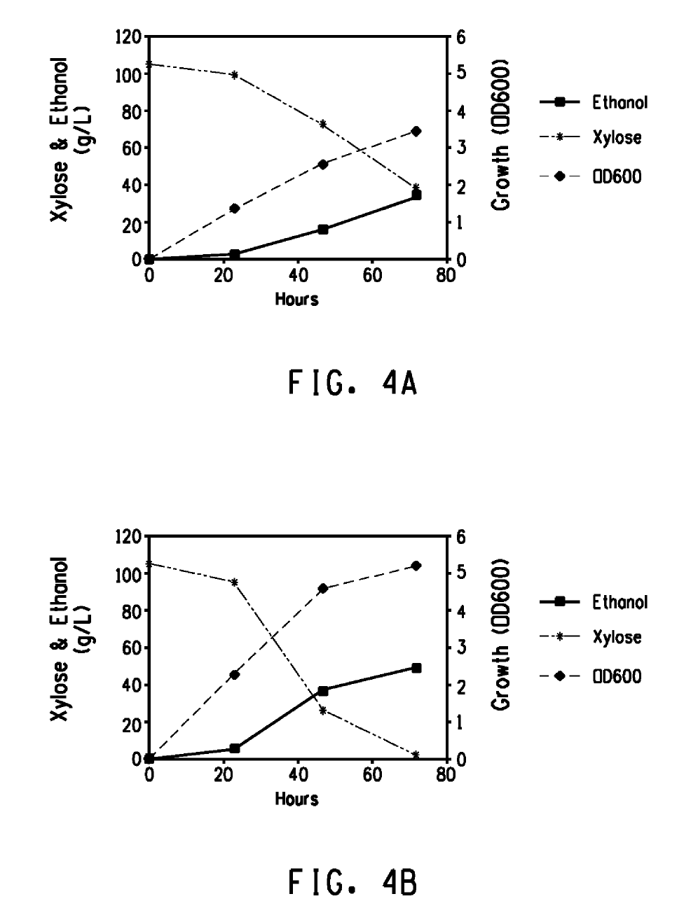

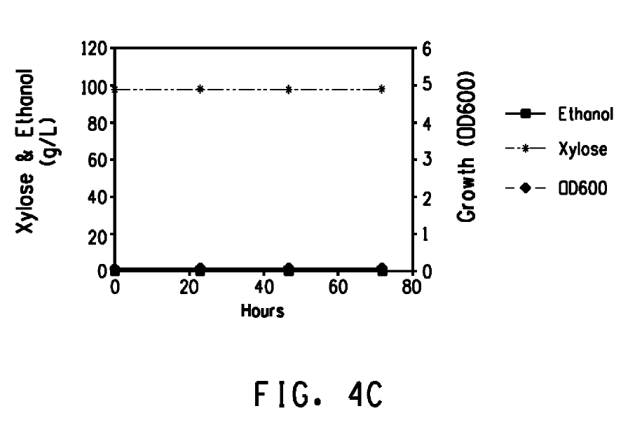

Shake flask fermentation was carried out in 20 mL mRM3-X10 in order to determine each strain's ability to use xylose. OD₆₀₀ value and both xylose and ethanol concentrations were measured at 0, 24, 48, and 72 hours. FIG. 4 is a summary of the results for ZW1-X109 (A), ZW1-210 (B) and ZW1 (C). The results confirm that both new strains were able to ferment xylose. After 72 hours of fermentation, ZW1-X109 had utilized approximately 64.2% of xylose (a reduction from 105.6 g/L to 37.8 g/L) to support an ethanol titer of 31.5 g/L and biomass growth to OD₆₀₀ value of 3.51; ZW1-X210 had utilized almost all available xylose (a reduction from 105.6 g/L to 1.6 g/L) to support an ethanol titer of 48.5 g/L and a biomass growth to OD₆₀₀ value of 5.22. However, ZW1 could not grow in mRM3-X10 due to lacking the xylose metabolic pathway. Therefore, among new strains, ZW1-X210 could ferment xylose faster than ZW1-X109, in the xylose-containing single sugar medium. The major difference between ZW1-X109 and ZW1-X210 is that the P_{gapT}-ECTal-ECTkt::Pₑₙₒ-ZMRpi-ZMRpe construct was inserted into the IdhA locus in ZW1-X109, and into the endogenous pnp gene in ZW1-X210. This result indicates that interruption of the pnp gene may benefit xylose metabolism in Z. mobilis.

The genomes of the ZW1-X109 and ZW1-X210 strains were sequenced and compared to the ZW1 genomic sequence as described in Example 1. Among the differences were sequence changes in the ZMO0976 coding region in both strains. In ZW1-X109 there was a G to A mutation at position 917 of the coding region sequence (in SEQ ID NO:1 native ZMO0976 seq). This mutation changed codon 306 from TGG (encoding tryptophan) to the TAG stop codon, which shortens the normally 340 amino acid encoded protein (SEQ ID NO:2) by eliminating 35 amino acids at the C-terminus. In the ZW1-X210 strain there is a large deletion including 125 nucleotides of the ZMO0976 promoter region and extending through 475 nucleotides of the coding region.

### Example 3

### Generation of a Suicide Construct to Knockout the ZMO0976 Gene in ZW1

To directly assess the effect of a ZMO0976 mutation alone on xylitol production in the wild type ZW1 strain, a suicide construct that can insertionally inactivate this gene was generated as described below.

Construction of pMODlinker-Cm

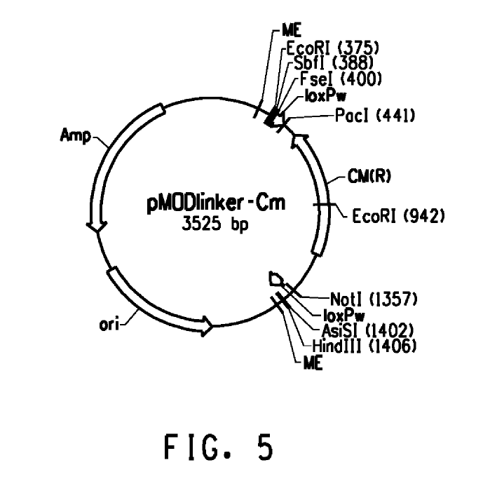

pMODlinker-Cm (FIG. 5) was an important plasmid intermediate in the generation of a suicide construct (pAR-cm) that was designed to insertionally-inactivate (“knock out”) the ZMO0976 gene (Seo et al (2005) Nature Biotechnol. 23:63-68) in Z. mobilis. A DNA fragment that confers resistance to chloramphenicol (Cm^{r}) was inserted between the NotI and PacI sites of the pMOD-Linker-Spec plasmid, which is described in detail in U.S. Pat. No. 7,989,206, replacing the DNA fragment that confers resistance to spectinomycin (Spec^{r}) as follows. pMOD-Linker-Spec was sequentially digested with PacI and NotI, and the 2.6 kb vector DNA fragment was purified from a 1% agarose gel. The Cm^{r} gene with its associated promoter was then PCR amplified from the commercially available plasmid pACYC184 (Boca Scientific, Boca Raton, Fla.), using primer Cm-F-NotI (SEQ ID NO. 44; NotI site underlined) and primer Cm-R-PacI (SEQ ID NO: 2; PacI site underlined).

Primer Cm-F-NotI (SEQ ID NO: 44):
CATCTTACTGCGGCCGCGTGACGGAAGATCACTTCGCAG
Primer Cm-R-PacI (SEQ ID NO: 45):
TCACTCATTTAATTAACTTATTCAGGCGTAGCACCAG

The 0.9 kb PCR product was also cut with PacI and NotI, and the purified DNA fragment was ligated to the PacI/NotI-cut vector fragment described above to yield pMODlinker-Cm (FIG. 5). This 3525 bp plasmid has a loxP-flanked, Cm^{r}-cassette that is located between the two mosaic ends (ME) that Tn5 transposase interacts with to form transposomes.
Construction of Plasmid pAR-cm

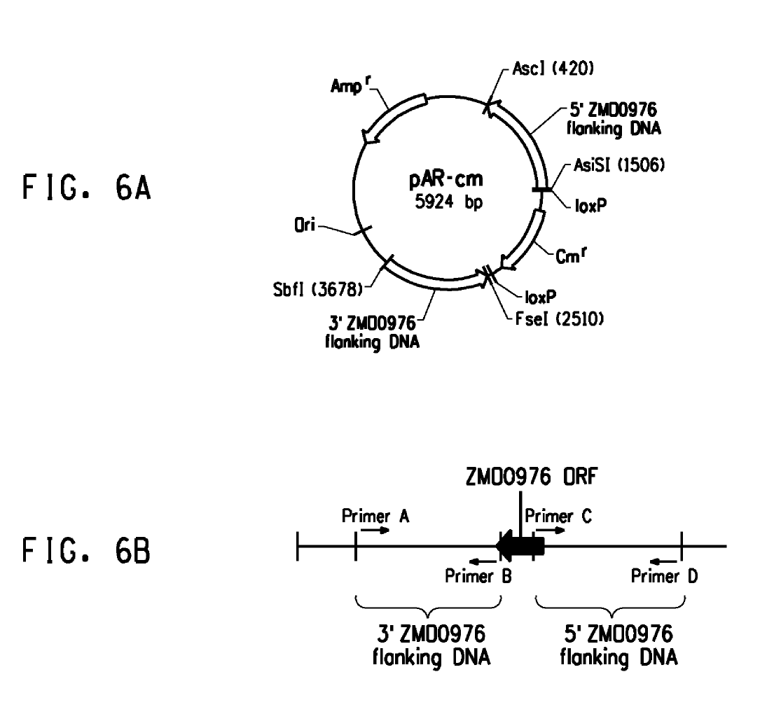

A suicide construct (pAR-cm) was used to knockout the ZMO0976 gene in ZW1, via host-mediated double-crossover homologous recombination. The plasmid that was used for this manipulation is analogous to the suicide construct (pHimA) that was previously used to knockout the himA gene in ZW801-4. The construction of pHimA was described in detail in U.S. Pat. No. 7,897,396, which is incorporated herein by reference. To generate the pAR-cm suicide construct (FIG. 6A) that was used in the present invention, the 5′- and 3′-himA flanking DNA fragments in pHimA were replaced with 5′- and 3′-ZMO0976 flanking DNA fragments to target the selectable marker and double-crossover event to the chromosomal ZMO0976 gene. Additionally, the loxP-flanked spectinomycin-resistance (Spec^{r}) cassette in pHimA was replaced with a loxP-flanked chloramphenicol-resistance (Cm^{r}) cassette. Four purified DNA fragments were required to the generate pAR-cm as described below.

Fragment 1 was obtained from pLDHSp-9WW that was previously described in U.S. Pat. No. 7,897,396 by cutting the plasmid with four different restriction enzymes: NotI, BsaI, SbfI and AscI. NotI cuts pLDHSp-9WW at nt 2647 and BsaI cuts the plasmid at nt 1816. After the plasmid DNA was completely digested with the four restriction enzymes, the 2666 bp SbfI-AscI DNA fragment was purified by electrophoresis using a 1% agarose gel and the Zymoclean Gel DNA Recovery Kit (catalog #D4001, Zymo Research). This fragment, named Fragment 1, contains an E. coli origin of replication that is not functional in Z. mobilis and a gene that confers ampicillin-resistance in E. coli.

Fragment 2 corresponds to the 1002 bp stretch of DNA that is located between the FseI and AsiSI sites in pMODlinker-Cm (FIG. 5). As indicated above, this DNA fragment contains a Cm^{r}-cassette that is flanked by two wild type loxP sites, one at each end. The complete DNA sequence of Fragment 2 is given in SEQ ID NO:46.

Fragment 3 contains 3′-ZMO0976 flanking DNA. This ˜1.2 Kb DNA fragment was generated by PCR using Primers A (SEQ ID NO:47) and B (SEQ ID NO:48). The template for PCR-amplification was genomic DNA that was isolated from ZW658 (ATCC #PTA-7858) using the Wizard Genomic DNA Purification Kit (catalog #A1125, Promega).

Primer A (SEQ ID NO: 47):
CTACTCATcctgcaggCTTCTCGGTGATCGTGTTGC
Primer B (SEQ ID NO: 48):
TCACTCATggccggccGAACAGATCGACGGTATTGATG

The underlined bases of Primer A (forward primer) binds downstream from the ZMO0976 coding region in the middle of a coding region for a hypothetical protein (product of the ZMO0975 gene), while the lower case letters correspond to an SbfI site that was added to the 5′ end of the primer. The underlined bases of Primer B (reverse primer) hybridize to the 3′ end of the ZMO0976 open reading frame, while the lower case letters correspond to an FseI site that was added to the 5′ end of the primer. The chromosomal binding sites for Primers A and B and the PCR product that was generated are shown in FIG. 6B. The PCR product was digested with SbfI and FseI, and the resulting 1168 bp fragment was then purified by agarose gel electrophoresis as described above.

Fragment 4 contains 5′-ZMO0976 flanking DNA. This ˜1.1 kb DNA fragment was generated by PCR using Primers C (SEQ ID NO:49) and D (SEQ ID NO:50). The template for PCR-amplification was genomic DNA that was isolated from ZW658 (ATCC #PTA-7858) using the Wizard Genomic DNA Purification Kit (catalog #A1125, Promega).

Primer C (SEQ ID NO: 49):
CATCTTACTgcgatcgcGATCAATCGCCCGATGAATG
Primer D (SEQ ID NO: 50):
CATCTTACTggcgcgccTCGCCGTATTGTATCGCTG

The underlined bases of Primer C (forward primer) hybridize at the 5′ end the of the ZMO0976 open reading frame, while the lower case letters correspond to an AsiSI site that was added to the 5′ end of the primer. The underlined bases of Primer D (reverse primer) hybridize upstream from the ZMO0976 gene in the middle of the open reading frame of a gene that codes for a putative “periplasmic binding protein”, while the lower case letters correspond to an AscI site that was added to the 5′ end of the primer. The chromosomal binding sites for Primers C and D and the PCR product that was generated are shown in FIG. 6B. The PCR product was digested with AsiSI and AscI, and the resulting 1086 bp fragment was purified by electrophoresis using a 1% agarose gel.

The four DNA fragments described above were then subjected to a 4-way ligation reaction to assemble the ZMO0976 knockout construct (pAR-cm), which is shown in FIG. 6A. The molar ratio of Fragments #1-#4 that was used for this reaction was approximately 1:1:1:1. An aliquot of the ligation reaction mixture was electroporated into E. coli DH10B and the transformed cells were plated on LB media that contained chloramphenicol (25 μg/ml). The plates were then incubated at 37° C. Chloramphenicol-resistant tranformants that contained plasmids with the correct size inserts were initially identified by colony PCR using Primer A/Primer D. Subsequent confirmation of positive clones came from DNA sequence analysis of the pAR-cm plasmid DNA from the PCR positive clones.

To obtain non-methylated plasmid DNA needed for transformation of Z. mobilis, pAR-cm was introduced into E. coli SCS110 (dcm⁻, dam⁻), and the transformed cells were plated on LB medium that contained chloramphenicol (25 μg/ml); growth was at 37° C. The chemically competent cells that were used for this manipulation were obtained from Stratagene (Cat. No. 200247) and the vendor's protocol was followed. It is important to note that the use of non-methylated plasmid DNA for transformation of Z. mobilis stains that are derived from ZM4 is critical for success, since methylated plasmid DNA that is isolated from wild type strains of E. coli, like DH10B, is readily destroyed by the host's restriction/modification systems. In the last step, plasmid DNA was isolated from one of the SCS110 transformants using the QIAGEN Plasmid Plus Midi Kit (Cat. No. 12943), and the final concentration of DNA was ˜1.5 μg/μl.

### Example 4

### Generation of the ZW1 ZMO0976 Knockout Mutant

To inactivate the ZMO0976 gene in ZW1, non-methylated pAR-cm plasmid DNA (which does not replicate in Z. mobilis) was introduced into ZW1 competent cells using electroporation as described in the GENERAL METHODS section. After a 3-hr recovery period in 1.0 ml MMG medium, the transformed cells were harvested by centrifugation (13,000×g for 1 min) and cell pellets were resuspended in 300 μL of MMG media. Aliquots of the cell suspension were then plated onto MMG agar plates that contained 120 μg/ml chloramphenicol (MMG/Cm120 plates), and the plates were incubated for 3 days at 33° C. under anaerobic conditions. Two of the resulting chloramphenicol-resistant colonies were randomly selected for further manipulation. Both colonies were streaked onto an MMG/Cm120 plate and incubated for 24 hr as described above. The new patches were then re-streaked onto a fresh MMG/Cm120 plate, and after a 24-hr growth period under the same conditions the resulting cell patches were subjected to further analysis as described below.

As described in U.S. Pat. No. 7,897,396, the initial interaction between the Z. mobilis chromosome and a suicide construct is a single-crossover event that takes place at one of the two flanking DNA sequences, and the single-crossover event eventually gives rise to double-crossover events. Transition to a double-crossover is normally very rapid (unless this event is lethal or results in a slower growth rate) and usually occurs after a few serial transfers in liquid or solid media that contains the selective agent for the suicide construct, in this case chloramphenicol. To confirm that the pAR-cm suicide construct had indeed undergone a double-crossover event at the correct location in the ZW1 chromosome in the two strains that were selected, colony PCR experiments were carried out using three different pairs of primers (Primer E/Primer F, Primer G/Primer H, Primer I/Primer J).

Primer E (SEQ ID NO: 51):
CTACTTCACTTCATGACCGG
Primer F (SEQ ID NO: 52):
AGTCATGCaggcctCTGATGAATGCTCATCCGGAA
Primer G (SEQ ID NO: 53):
GTCTGACGTTGATCCTGATC
Primer H (SEQ ID NO: 54):
TCACTCATggccggccTGCGTATAATATTTGCCCATGG
Primer I (SEQ ID NO: 55):
GTTCCTGCTTTGCTTTTGTGG
Primer J (SEQ ID NO: 56):
CCCGGAAGCTATCAAAATTTTG

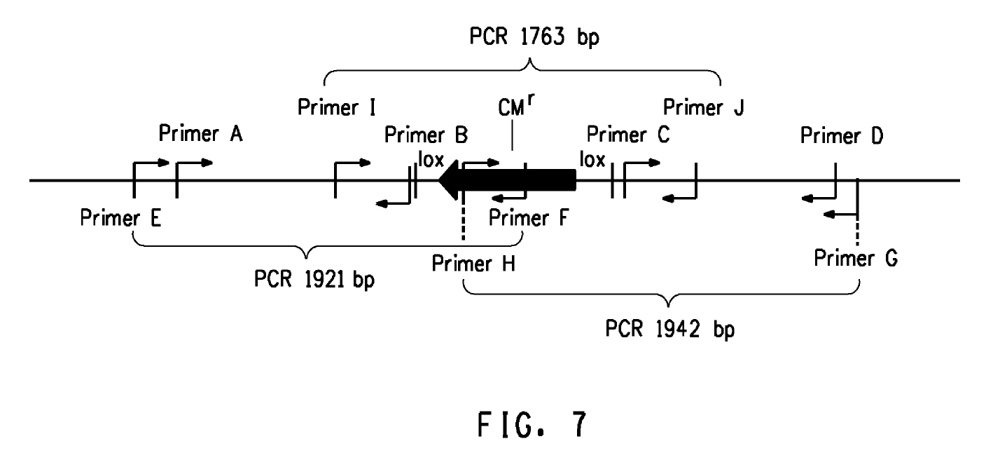

Primers E, I, J, and G hybridize to the Z. mobilis chromosome at the locations shown in FIG. 7. The underlined bases of Primer F and H hybridize to the chloramphenicol-resistance (Cm^{r}) cassette that is inserted into the chromosome after the desired double-crossover event has occurred (FIG. 7). A 1921 by PCR product using Primer E and F indicates that the correct single-crossover event occurred at one end of the ZMO0976 gene, while a 1942 bp PCR product using Primer G and H indicates that the correct single-crossover event occurred at other end. Primers I and J can only generate a single 1763 bp PCR product if the correct double-crossover event has occurred. In contrast, this pair of primers would generate a 1468 bp PCR product with wild type ZW1 strain or a mixed population of transformants that had not yet completed the transition from the single-crossover event to the double-crossover event. The presence of the 1763 bp PCR product and the absence of the 1468 bp PCR product with Primers I and J indicate a homogeneous population of ZMO0976 knockout mutants that have the desired double crossover event. Using the PCR strategy described above, both colonies that were selected for further analysis were shown to be true double-crossover events, and these two strains were named AR1 and AR2.

### Example 5

### Effect of Insertional-Inactivation of ZMO0976 on Xylitol Production In Vivo and NADPH-Dependent Xylose Reductase Activity in Cell-Free Extracts

To test the possibility that the wild type ZMO976 gene encodes an enzyme that is able to convert D-xylose to xylitol in living cells, xylitol formation was monitored for wild type ZW1 and the two ZW1/ZMO0976 knockout mutants (AR1 and AR2) during growth in a synthetic medium that contained both glucose and xylose. The seed cultures for this experiment were grown at 33° C. in 7 ml of 80 g/L glucose, 10 g/L yeast extract, 2 g/L KH₂PO₄, and 1 g/L MgSO₂(7H₂O) to an OD600 of ˜1.8. Aliquots of the seed cultures were then used to inoculate 10-ml cultures that contained 60 g/L glucose, 20 g/L xylose, 10 g/L yeast extract, 6 g/L KH₂PO₄, 1 g/L MgSO₂(7H₂O), and 2.5 g/L (NH₄)₂SO₄, the final pH was adjusted to pH 5.9 with concentrated potassium hydroxide. The cultures were grown at 33° C. and the initial OD₆₀₀ values were ˜0.11. After 0, 15.5, 39, 60, and 112 hours of growth, 1.0-ml aliquots of the cultures were removed for HPLC analysis using an HP 1100 equipped with a refractive index detector (Hewlett-Packard, Palo Alto, Calif.) to determine the concentration of xylitol that was present in the fermentation broth, using the methodology described in the General Methods section.

As shown in FIG. 8, the rate and extent of xylitol production were much greater for wild type ZW1 than they were for the two ZW1/ZMO0976 knockout mutants. For example, during the first 40 hours of the experiment the amount of xylitol in the fermentation broth for both AR1 and AR2 was below the level of detection for the HPLC, which is ˜0.1 g/L. And by the end of the experiment wild type ZW1 had generated ˜3- to 4-fold more xylitol than either of the ZW1/ZMO0976 knockout mutants; 0.736 g/L versus 0.254 g/L and 0.19 g/L for AR1 and AR2, respectively. It should be noted that the lower amounts of xylitol produced by AR1 and AR2 were not a consequence of lower cell density: the OD600 values for AR1 and AR2 at the 39-hr time point after all the glucose had been consumed were 7.29 and 6.98 compared to 6.25 for wild type ZW1.

The amount of xylitol generated by wild type ZW1 in the above experiment was similar to values previously reported for the same strain harboring the control plasmid pZB188/Kan under comparable experimental conditions (FIG. 14 A in U.S. Pat. No. 7,741,084). However these values are at least 3-fold lower than the amount of xylitol that was generated when the xylose isomerase expression plasmid, pZB188/Kan-XylA, was introduced into wild type ZW1 (FIG. 14 B in U.S. Pat. No. 7,741,084). The larger amounts of xylitol that are observed when xylose can be converted to xylulose by xylose isomerase is the consequence of glucose-fructose oxidoreductase (GFOR), which in turn is then able to convert xylulose to xylitol. As described in U.S. Pat. No. 7,741,084, GFOR is the major contributor to xylitol formation in vivo, but only when glucose is present and a source of xylulose is available. The results in FIG. 8 clearly demonstrate that the ZMO0976 gene product is also a significant contributor to xylitol production in vivo. However, it is also evident that inactivation of this gene does not entirely eliminate xylitol production in vivo, even under conditions where the GFOR-mediated route for xylitol is inoperative due to the absence of xylose isomerase in the wild type strain, ZW1. To provide a direct demonstration that the ZMO0976 gene product is able to catalyze the conversion of xylose to xylitol, cell-free extracts were prepared for wild type ZW1 and for AR1 and NADPH-dependent xylose reductase activities were measured. Preparation of the cell-free extracts and the spectrophotometric enzyme assay that was used for this experiment are described in the General Methods section. The specific activity of NADPH-dependent xylose reductase activity for wild type ZW1 was 14 mU/mg under the conditions employed. In marked contrast, NADPH-dependent xylose reductase activity was not detectable for the ZW1/ZMO0976 knockout mutant. Indeed, the rate of NADPH disappearance in the cell-free extract prepared from the AR1 strain was slightly faster in the absence of xylose than in the presence of xylose. Thus it is safe to conclude from this experiment that elimination of the ZMO0976 gene product in ZW1 reduced NADPH-dependent xylose reductase activity by greater than 90%.

### Example 6

### Inactivation of ZMO0976 Allows Immediate Growth on Xylose without Adaptation and Direct Selection on Xylose for a Xylose-Utilization Pathway

To test the hypothesis that a functional ZMO0976 gene constitutes a non-permissive condition for growth on xylose for recombinant strains of Z. mobilis that harbor the four genes that are required for xylose metabolism, plasmid pZB4 was introduced into wild type ZW1 and the ZW11ZMO976 knockout mutant, AR1. As described in U.S. Pat. No. 5,514,583 Example 3, which is incorporated herein by reference, pZB4 is a multi-copy shuttle vector that replicates in E. coli and Z. mobilis. In addition to a tetracycline-resistance cassette that serves as the selectable marker, this plasmid contains the four genes that are needed to complete a xylose metabolic pathway for Zymomonas, which are expressed from two synthetic chimeric operons. One of the operons contains the E. coli xylose isomerase and xylulokinase coding regions under the control of the Z. mobilis Pgap promoter (P_{gap}-xylA/xylB operon), while the other consists of the E. coli transketolase and transaldolase coding regions that are driven by the Z. mobilis Peno promoter (P_{ENO}-tal/tkt operon).

Non-methylated pZB4 plasmid DNA was electroporated into ZW1 and AR1 as described in the General Methods section. After electroporation the entire 50-μl transformation reaction was added to 10 ml of MMG medium and the cells were allowed to recover for 5 hrs at 30° C. under static conditions. The cells were then harvested by centrifugation and 8 ml of the supernatant was removed and replaced with an equal volume of MMG medium (without MgSO₄) that contained 25 μg/ml of tetracycline. The resulting cultures were then incubated for ˜18 hrs at 33° C. to enrich for plasmid-bearing transformants. This enrichment step is very important since the transformation efficiency of pZB4 is extremely low, presumably due to the large size of this plasmid. Following the enrichment process the cells were collected by centrifugation and the cell pellets were resuspended in 800 μl of MMG (without MgSO₄). Next, a 100-μl aliquot of each cell suspension was plated onto an MMG agar plate (without MgSO₄) that contained tetracycline (20 μg/ml), and the plates were incubated at 33° C. in an anaerobic chamber. After 48 hrs there were ˜100 colonies on the plates for both the ZW1 and AR1 transformants and they were of similar size (2-3 mm) for both strains. Three ZW1/pZB4 colonies (-A, -B and -C) and three AR1/pZB4 colonies (-1, -2 and -3) were then randomly chosen for further characterization in the experiment described below without any additional manipulations. That all six strains harbored the pZB4 plasmid was confirmed by PCR analysis of resuspended cells using Primer TF-2 and Primer TR-9 (SEQ ID NOs:57 and 58). These oligonucleotides hybridize to the Peno-Tal/Tkt operon that is present in pZB4 and amplify a DNA fragment that is 1681 bp, and all six strains produced a DNA fragment with the correct size.

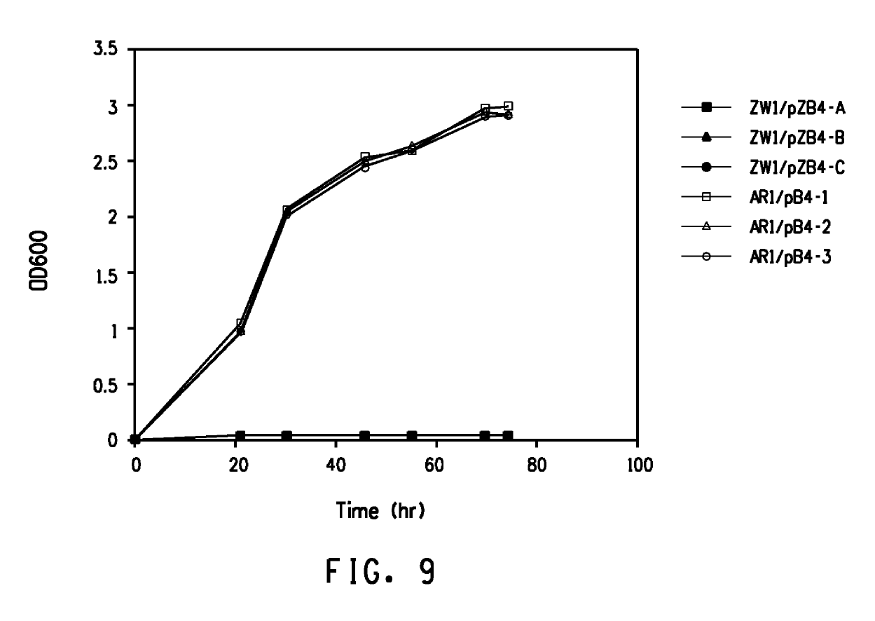

FIG. 9 shows a shake flask experiment using mRM3-X10 (which contains 100 g/L xylose as the only sugar) plus 20 μg/ml of tetracycline as the test medium. The inocula used for this study were the six colonies from the glucose/tetracycline agar plates that were described above, which previously had not been exposed to xylose. Cultures were grown at 33° C. and the initial OD600 was ˜0.035. The three ZW1/pZB4 transformants that had a wild type ZMO0976 gene failed to grow on xylose despite the fact they contain all four genes that are required for xylose metabolism (FIG. 9). Indeed, even after a 3-day incubation period the OD600 of these cultures only increased about 60%, which constitutes less than one doubling. In striking contrast, when the three ZW1/ZMO976 knockout mutant colonies that harbored the pZB4 plasmid were transferred from the MMG/tetracycline plate into xylose-containing medium, growth began immediately without the need for a preliminary xylose-adaptation step (FIG. 9).

Given the above results, experiments were performed to test whether it was possible to recover AR1/pZB4 transformants by plating them directly onto solid medium that contained xylose as the sole carbon source, without tetracycline. The pZB4 plasmid DNA was introduced into ZW1 and AR1 using the same protocol that is described above including the 18-hr enrichment step with tetracycline. Following this procedure, the cells were harvested by centrifugation, washed twice with 1.0 ml of MMX medium (same as MMG medium but contains 50 g/L xylose instead of glucose) and finally resuspended in 800 μl of the same medium. One hundred microliter aliquots of the resulting cell suspensions were then spread onto an MMX agar plate and an MMG agar plate (without MgSO₄) that contained tetracycline (20 μg/ml), and the plates were incubated at 33° C. under anaerobic conditions. After a 24-hr incubation period there were 132 and 118 colonies on the MMG/tetracycline plates for ZW1 and AR1, respectively. Thus the transformation efficiency with the pZB4 plasmid DNA was virtually identical for both strains. However, very different results were obtained when the same cell suspensions were plated directly onto MMX agar plates lacking tetracycline: After a 100-hr incubation period there were 85 colonies (˜1 mm) for AR1 and no colonies for the ZW1 parent strain that has a wild type ZMO0976 gene.

The above results clearly demonstrate that a functional ZMO0976 gene product presents a major obstacle for growth on xylose for recombinant strains of Z. mobilis that are genetically engineered for xylose metabolism. This enzyme is an NADPH-dependent aldose reductase that is able to convert xylose to xylitol. Although xylitol per se does not inhibit bacterial growth or cause cell death it is a well-known alternate substrate for E. coli xylulokinase, which phosphorylates it in the presence of ATP to form the toxic compound xylitol 5-phosphate. However, as shown in FIG. 8, inactivation of the ZMO0976 gene resulted in a 3- to 4-fold reduction in xylitol formation in vivo, and this allowed the AR1/pZB4 transformants to immediately grow on xylose when a xylose-utilization pathway was introduced. In other words, the AR1/pZB4 transformants are able to grow on xylose without an adaptation step because they generate less xylitol and hence form less xylitol 5-phosphate than the ZW1/pZB4 transformants. The experiment described above further demonstrates that inactivation of the ZMO0976 gene can allow direct selection on xylose, provided the xylose pathway enzymes that are introduced can provide sufficient carbon flux to support bacterial growth when xylose is the only carbon source available.

### Example 7

### Adaptation of a ZW1/pZB4 Transformant Results in at Least Three Different Types of Mutations

As shown in FIG. 9, the ZW1/pZB4-A transformant that was obtained through tetracycline-selection on a glucose plate did not grow on xylose when it was inoculated into mRM3-X10 medium that contained 20 μg/ml tetracycline. Growth was also not observed when this strain was inoculated into MMX10 medium (same as MMX medium but contains 100 g/L xylose instead of 50 g/L xylose) that lacked tetracycline. Indeed, the latter experiment was performed eight different times using an initial OD of ˜0.2, and there was no increase in turbidity for any of the cultures even after a 6-day incubation period at 33° C. However, we were able to adapt the ZW1/pZB4-A strain for growth on xylose by using a mixture of glucose and xylose as described in more detail below.

ZW1/pZB4-A glycerol stock was inoculated into 10-ml of mRM3-G5 medium that contained 20 μg/ml tetracycline and the initial OD was 0.1; mRM3-G5 is identical to mMR3-G10 but contains 50 g/l glucose instead of 100 g/L. After a 9-hr incubation period at 33° C. the OD increased to ˜2.0, and the cells were collected by centrifugation. The cell pellet was resuspended in 250 ml of 45 g/L xylose, 5 g/L glucose, 10 g/L yeast extract, 5 g/L of tryptone, 2.5 g/L of (NH₄)₂SO₄, 0.2 g/L K₂HPO₄, and 0.1 mM MgSO₄ and 20 μg/ml tetracycline to an initial OD of 0.08. Twenty three milliliter aliquots of the resuspended cells were then distributed to eight 50-ml loosely capped test tubes and the cultures were gently shaken at 33° C. for 86 hrs. During the incubation period the OD values increased to ˜1.0, and an aliquot of each culture was then added to 5 ml of mRM3-X10 medium. The new cultures were incubated at 33° C. for 28.5 hrs during which time their OD values increased from ˜0.04 to 0.7-1.1. Aliquots from each of the eight cultures were then individually plated onto an MMG agar plate (without MgSO₄) that contained 20 μg/ml tetracycline to isolate single colonies. After a 2-day incubation period at 33° C., two colonies from each plate were randomly selected for further characterization; these colonies were named after the original eight cultures and are referred to below as strains 1A, 1B, 2A, 2B, 3A, 3B, 4A, 4B, 5A, 5B, 6A, 6B, 7A, 7B, 8A and 8B.

A preliminary experiment was performed to see how well the various strains grew on xylose. Each of the 16 colonies was separately inoculated into 5 ml of mRM3-X10 media and the cultures were incubated for 21.5 hrs at 33° C. Based on the extent of growth during this experiment (increase in OD), one colony from each plate was chosen for further characterization and it was the one that grew the best in xylose. The selected strains were 1A, 2B, 3B, 5B, 6A, 6B, 7A and 8B, representing seven of the eight original cultures. Since strains 4A and 4B barely grew on xylose during the preliminary experiment, they were not included in the study described below, and both 6A and 6B were included.

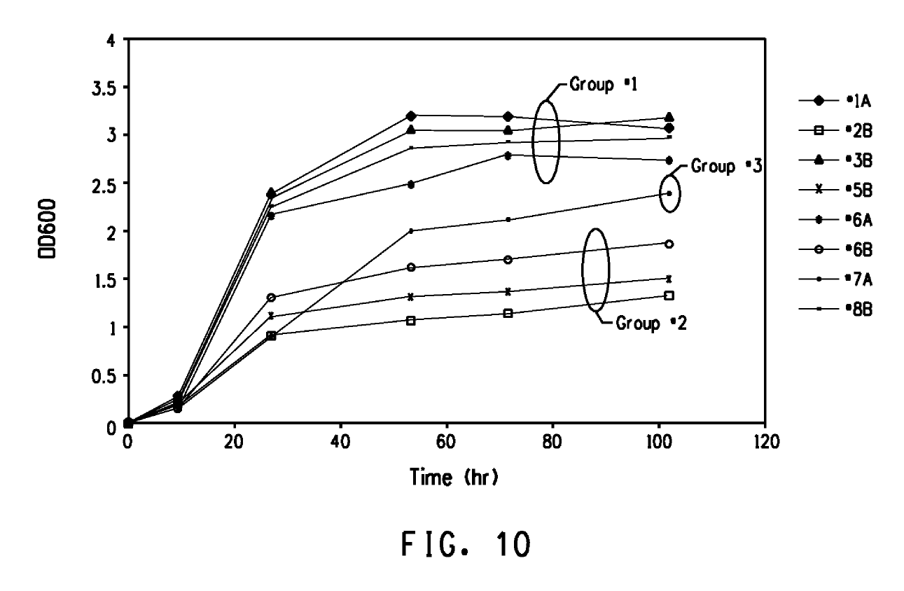

A shake flask experiment with mRM3-X10 as the growth medium was performed using the eight adapted strains that were selected above. The initial OD of the cultures was ˜0.05 and the temperature was 33° C.; the pre-cultures that were used as inocula for this experiment were also grown in mRM3-X10. Based on growth in xylose and HPLC analysis of the fermentation broth the strains appeared to fall into three different groups as shown in FIG. 10. Group #1 strains (1A, 3B, 6A and 8B) all behaved similarly and grew faster on xylose than the other four strains. They also grew to a higher final OD. Group #2 strains (2B, 5B and 6B) also grew with similar kinetics, but their growth rates and final OD values were much lower than Group #1 strains. Although the Group #3 strain (7A) was the slowest grower, it eventually reached a higher OD than the Group #2 strains.

Table 1 shows endpoint values (102 hrs) for xylose, xylulose, ribulose, xylitol, glycerol, acetate and ethanol in the fermentation broth of the eight cultures described above as determined by HPLC analysis. Note that all four Group #1 strains generated large amounts of xylitol (>3 g/L), and this was also true for the slow growing Group #3 strain, 7A. In marked contrast, the amount of xylitol that was present in the fermentation broth for strains 2B, 5B and 6B was below the level of detection and could not be quantified. The three groups also had different patterns and levels for other by-products apart from xylitol. For example, the Group #1 and Group #3 strains generated significant amounts of acetate, but relatively low levels of ribulose and glycerol. On the other hand, the Group #2 strains produced large amounts of ribulose and glycerol, but barely made any acetate. Finally, only the Group #3 strain (7A) produced a large amount of xylulose (˜4 g/L) which is the natural substrate for the second enzyme in the engineered xylose pathway, namely xylulokinase. It was also interesting that although the Group #1 strains grew faster in xylose and to higher final ODs than the Group #2 strains, their rates of ethanol production were significantly slower than the other two groups.

TABLE 1
Fermentation broth analysis of strains adapted for growth in
xylose-containing medium.

| | | | | | | | | | E. coli |
|---|---|---|---|---|---|---|---|---|---|
| | | | | | | | | ZM0976 | XylB |
| Strain | Xylose | Xylulose | Ribulose | Xylitol | Gycerol | Acetate | EtOH | Mutation | Mutation |
| Group | | | | | | | | | |
| #1 | | | | | | | | | |
| #1A | 55.06 | 0 | 0.99 | 3.39 | 0.68 | 0.74 | 18.55 | NO | YES |
| #3B | 56.52 | 0 | 0.97 | 3.26 | 0.69 | 0.74 | 17.08 | NO | YES |
| #6A | 56.95 | 0 | 1.05 | 2.98 | 0.75 | 0.49 | 18.15 | NO | YES |
| #8B | 56.30 | 0 | 0.94 | 3.25 | 0.69 | 0.71 | 17.72 | NO | YES |
| Group | | | | | | | | | |
| #2 | | | | | | | | | |
| #2B | 46.64 | 0 | 4.08 | 0 | 3.19 | 0 | 21.59 | YES | NO |
| #5B | 46.10 | 0 | 4.45 | 0 | 3.63 | 0 | 21.73 | YES | NO |
| #6B | 41.13 | 0 | 4.50 | 0 | 3.56 | 0 | 23.25 | YES | NO |
| Group | | | | | | | | | |
| #3 | | | | | | | | | |
| #7A | 49.79 | 3.97 | 0 | 4.53 | 1.06 | 1.07 | 19.06 | NO | *nd |

*nd = not determined

To determine whether any of the eight adapted strains had mutations in ZMO0976, the gene was PCR-amplified using Primer I and Primer G (SEQ ID NOS:55 and 53) and resuspended cells as a template. The DNA fragment that was generated contained the entire ZMO0976 coding region plus ˜900 nucleotides upstream from the start codon and ˜200 nucleotides downstream from the stop codon. The resulting PCR products were subjected to DNA sequence analysis using six different primers (SEQ ID NOs:59-64).

Only the Group #2 strains had a mutation in the ZMO0976 gene. Strain #5B had an 8-bp deletion in the open reading frame that resulted in a frame shift. The start of the deletion was 858 nucleotides downstream from the first nucleotide of the initiation codon. The two other Group #2 strains (2B and 6B) were identical siblings that had a G to T point mutation in the open reading frame at nucleotide 349 that converted amino acid residue E117 to a stop codon. These results are consistent with the HPLC data that is shown in Table 1, since the Group #2 strains were the ones that generated only trace amounts of xylitol.

The large amounts of xylitol that were found in the fermentation broth for the Group #1 and Group #3 strains strongly suggested that these strains had evolved a different mechanism for coping with xylitol toxicity. As already indicated it is xylitol 5-phosphate, not xylitol, that is responsible for the inhibitory effects on Z. mobilis growth and viability, and the former compound is generated by E. coli xylulokinase in a reaction that requires ATP and xylitol. It thus seemed possible that the Group #1 and Group #3 strains had acquired mutations in the E. coli xylulokinase gene (xylB), which is present in the pZB4 plasmid DNA that was introduced into these strains. To test this hypothesis the E. coli xylB gene (NCBI accession number NC_{—}000913, Gene ID: 948133) was amplified from the eight adapted strains using two PCR primers (SEQ ID NOs:65 and 66) and resuspended cells as a template.

The DNA fragment that was generated contained the entire xylB coding region and ˜300 nucleotides downstream from the stop codon. The resulting PCR products were subjected to DNA sequence analysis using six different primers (SEQ ID NOs:67-72). Sequencing results showed that all of the Group #1 strains (1A, 3B, 6A and 8B) had a point mutation in the E. coli xylulokinase open reading frame, and it was the exact same modification indicating they were siblings. All four strains had a G to A replacement at nucleotide 224 (relative to the first nucleotide of the start codon) that resulted in a single amino acid substitution, a G75D mutation. Presumably this mutation either lowers the specific activity of xylulokinase or reduces its substrate specificity towards xylitol. Either scenario would result in the formation of less xylulose 5-phosphate, allowing these strains to grow in xylose during on-going ZMO0976-mediated xylitol production.

To determine the effect of the xylB point mutation on xylulokinase enzyme activity, cell-free extracts were prepared for one of the Group #1 strains (3B) and one of the unadapted ZW1/pZB4 strains (ZW1/pZB4-B); the latter has a wild type xylulokinase gene. The extracts were prepared essentially as described in the General Methods section but the cells were harvested at an OD of ˜1.0. The resulting extracts were used to measure xylulokinase activity in an enzyme-coupled reaction with lactate dehydrogenase and pyruvate kinase. The 1.0-ml reactions were conducted in a quartz cuvette at 20° C. and contained the following components: 50 mM Tris-HCl (pH 7.5), 5 mM MgCl₂, 50 mM KCl, 1 mM ATP, 1 mM EDTA, 1 mM DTT, 20 units of lactate dehydrogenase, 80 units of pyruvate kinase, 1.5 mM phosphoenolpyruvate, 0.2 mM NADH, 5 mM D-xylulose and the equivalent of 2.5-10 μl of cell-free extract. The xylulose was added last after establishing baseline conditions. Control reactions were identical, but no xylulose was added. Enzyme activity was measured spectrophotometrically as a function of time at 340 nm using an extinction coefficient of 6220 M⁻¹cm⁻¹ to monitor the conversion of NADH to NAD. Enzyme activities are expressed as U/mg of cell-free extract protein after correcting for the rate of NADH oxidation in the absence of xylulose; 1 U=1 micromole of xylulose 5-phophate formed per minute. The signal-to-noise ratio for xylulose-dependent NADH oxidation was greater than 60 when 10 μl of cell-free extract from the unadapted ZW1/pZB4 strain was used for the assay (i.e. the rate of NADH disappearance in the presence of xylulose compared to the control reaction).

The average xylulokinase specific activity for the unadapted ZW1/pZB4 strain was 3.67 U/mg under the conditions employed, based on three replicate determinations that varied by less than 7%. In marked contrast, the mean specific activity for the adapted 3B strain was only 0.21 U/mg, based on duplicate determinations that varied by 16%. Thus the G75D point mutation in the adapted strain 3B reduced the specific activity of E. coli xylukinase by ˜95% compared to the wild type gene. Although this mutation clearly allows the cells to survive during ongoing ZMO0916-mediated xylitol, it is clear from the xylose shake flask experiments that are shown in FIG. 10 that it also has a detrimental effect on the rates of xylose utilization and ethanol production. More important, all four strains that have this mutation generate significant amounts of xylitol (Table I) which greatly reduces the metabolic yield for ethanol production.

The nature of the mutation for the Group 3 strain, 7A, remains to be determined. Although there were no mutations in the plasmid-born E. coli xylB open reading frame, the data shown in Table I and FIG. 10 strongly suggests that this adapted strain also has a very low level of xylulokinase enzyme activity. Indeed, in addition to accumulating large amounts of xylitol, 7A was the only strain that produced detectable amounts of xylulose, indicating that E. coli xylulokinase was not able to phosphorylate xylulose as fast as xylose isomerase could produce it.

In summary, inactivation of the ZMO0976 gene greatly reduces xylitol production and the accumulation of toxic xylitol 5-phosphate. This strategy also eliminates the need for a preliminary adaptation step and allows immediate growth on xylose for recombinant Z. mobilis strains that have a xylose metabolism pathway. More important, intentional inactivation of ZMO0976 minimizes the possibility of acquiring other spontaneous mutations that can occur during the xylose adaptation process that help the organism cope with xylitol toxicity, such as the E. coli xylB mutation described above.

What is claimed is:

1. A method of making a xylose-competent Zymomonas cell comprising:
a) providing a Zymomonas host cell;
b) creating a genetic modification in said cell in at least one endogenous gene encoding an aldose reductase enzyme of EC 1.1.1.21 having the ability to convert xylose to xylitol in the presence of NADPH; and
c) introducing into said cell a xylose utilization metabolic pathway;
wherein the order of steps (b) and (c) is not specified and wherein said xylose-competent Zymomonas cell has aldose reductase activity reduced by greater than 90% as compared with a Zymomonas cell lacking the genetic modification of step (b).

2. The method of claim 1 further comprising selecting the xylose-competent Zymomonas cell using medium containing xylose as the only sugar.

3. The method of claim 1 wherein the Zymomonas host cell is a wild type cell.

4. The method of claim 1 wherein the Zymomonas host cell is a Zymomonas cell that has not previously been exposed to xylose.

5. The method of claim 1 wherein steps (b) and (c) occur concurrently

6. The method of claim 1 wherein the endogenous gene encoding aldose reductase encodes a protein having at least about 95% sequence identity to the amino acid sequence as set forth in SEQ ID NO:2.

7. The method of claim 1 wherein the endogenous gene encoding aldose reductase has a coding region sequence having at least about 95% identity to the nucleotide sequence as set forth in SEQ ID NO:1.

8. The method of claim 1 wherein the genetic modification of step (b) is selected from the group consisting of mutation, insertion, deletion, and combinations thereof.

9. The method of claim 1 wherein the xylose utilization metabolic pathway comprises a series of polynucleotides encoding polypeptides, each having xylose isomerase, xylulokinase, transketolase, or transaldolase enzymatic activity.

10. The method of claim 9 wherein the polypeptide having xylose isomerase activity is a Group I xylose isomerase and is included in the class of enzymes identified by EC 5.3.1.5.

11. The method of claim 10 wherein the polynucleotide encoding the xylose isomerase polypeptide is isolated from Actinoplanes missouriensis

12. The method of claim 11 wherein the polynucleotide encoding the xylose isomerase polypeptide is operably linked to a mutant glyceraldehyde-3-phosphate dehydrogenase gene promoter, wherein the mutant promoter has higher activity than the native promoter.

13. The method of claim 1 wherein the xylose-competent Zymomonas cell further comprises at least one genetic modification which reduces glucose-fructose oxidoreductase activity.

14. The method of claim 1 wherein the xylose-competent Zymomonas cell further comprises a genetic modification which reduces expression of the endogenous himA gene.

15. The method of claim 1 wherein the xylose-competent Zymomonas cell further comprises polynucleotides encoding polypeptides for arabinose utilization, each having L-arabinose isomerase activity, L-ribulokinase activity, or L-ribulose-5-phosphate-4-epimerase activity, and optionally a polynucleotide encoding a polypeptide that is an arabinose-proton symporter.

16. The method of claim 1 wherein the xylose-competent Zymomonas cell further comprises a genetic modification which increases ribose-5-phosphate isomerase activity.

17. The method of claim 1 further comprising adapting the xylose-competent Zymomonas cell in xylose-containing medium wherein xylose utilization is improved.

18. A xylose-competent and ethanol-producing Zymomonas cell having the following characteristics:
a) the cell does not require a xylose-adaptation step for immediate growth on media containing xylose as the only sugar;
b) the cell has aldose reductase activity for conversion of xylose to xylitol in the presence of NADPH reduced by greater than 90%; and
c) the cell comprises a xylose utilization metabolic pathway comprising a series of polynucleotides encoding polypeptides, each having xylose isomerase, xylulokinase, transketolase, or transaldolase enzymatic activity.

19. A method for producing ethanol comprising growing the xylose-competent and ethanol-producing Zymomonas cell of claim 18 under conditions wherein ethanol is produced.
